(19) United States
(12) Patent Application Publication
(10) Pub. No.: US 2020/0199038 A1
(43) Pub. Date: Jun. 25, 2020

(54) DICARBOXYLIC FATTY ACID DIMERS, AND DERIVATIVES THEREOF, AS STANDARDS FOR QUANTIFYING LEVELS IN BIOSPECIMENS

(71) Applicant: Med-Life Discoveries LP, Saskatoon, Saskatchewan (CA)

(72) Inventors: Dushmanthi Jayasinghe, Saskatoon (CA); Shawn Ritchie, Saskatoon (CA); Daniel E. Levy, San Mateo, CA (US)

(21) Appl. No.: 16/622,363

(22) PCT Filed: Jun. 15, 2018
(86) PCT No.: PCT/CA2018/050729

§ 371 Date: Dec. 13, 2019

Related U.S. Application Data
(60) Provisional application No. 62/520,292, filed on Jun. 15, 2017.

(51) Int. Cl. C07B 59/00; C07C 57/13; C07F 7/18; G01N 33/534; G01N 33/574; G01N 33/92

(52) U.S. Cl. CPC C07B 59/001; C07C 57/13; C07F 7/1804; C07B 2200/05; G01N 33/57419; G01N 33/92; G01N 33/534

(57) ABSTRACT

A gastric tract acid (GTA) compound having the structure of formula I, as well as salts, esters, prodrugs, or labelled derivatives thereof, are provided. Such GTA compounds may be used for determining GTA levels of a sample, for diagnosing a subject as having or being at risk of developing colorectal cancer, or for raising antibodies. Antibodies, or fragments thereof, which specifically bind to the GTA of formula I are described, as well as uses of such antibodies or fragments for determining GTA levels in a sample, or for diagnosing a subject as having or being at risk of developing colorectal cancer. Kits comprising such GTA compounds and/or antibodies are also provided.

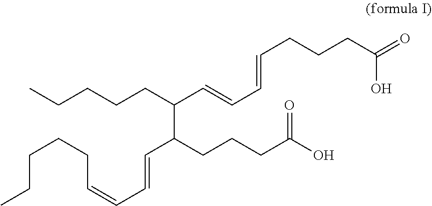 [SMILES: CCCCC/C=C\C=C\C(CCCC(=O)O)C(/C=C/C=C/CCCC(=O)O)CCCCC]

### FIELD OF INVENTION

The present invention relates generally to dicarboxylic acids and derivatives thereof. More specifically, the present invention relates to dicarboxylic acid compounds, and labelled derivatives thereof, for use in diagnosis of colorectal cancer.

### BACKGROUND

Gastric tract acids (GTAs) were initially discovered and reported as 28 to 36 carbon polyunsaturated fatty acids that were reduced in the serum of colorectal cancer (CRC) patients relative to control subjects. A commercial screening assay, (Cologic®), based on measuring a specific 28-carbon GTA called GTA-446, was developed for screening the average-risk population for increased risk of CRC.

The Cologic® test has been previously performed using tandem mass spectrometry to quantify the amount of GTA-446 in a sample by extrapolating the signal response of select parent/daughter fragment pair(s) of GTA-446 from an external GTA-446 standard curve comprised of serially diluted known concentrations of GTA-446. However, due to unavailability of commercially available synthetic GTA-446, the calibration curve relies on GTA-446 purified from large quantities of human serum (typically 50 liters or more). A disadvantage of this approach is that the isolation process is labour-intensive, yields only a small quantity (˜5 mg from 40 L of human serum), and only provides an endogenous, naturally-occurring species. Certain assay limitations therefore arise, because there has been no option for, for example, a labelled internal standard to control for recovery and other potential sources of variability such as, for example, matrix effect and/or drift in instrument sensitivity.

Typically, analytical approaches favor the use of internal standards in such applications. A favoured analytical approach would be to spike a known quantity of a stable isotope-labelled version of the analyte of interest into the sample being tested, and then determine the ratio of the endogenous analyte to the labelled standard. This ratio may then be used to extrapolate a quantitative assessment of the target analyte in the sample.

In addition to being difficult to obtain even in small quantities, the gastric tract acid GTA-446 has not been fully characterized, has not been synthetically prepared, and labelled derivatives have not been generated. Furthermore, until now, GTA-446 (C₂₈H₄₆O₄) was thought to be a single long-chain fatty acid containing four unsaturations, a single carboxylic acid moiety, and two hydroxy moieties.

The quantification of GTA-446 has previously been further limited to primarily tandem mass spectrometry analyses, since enzyme-linked immunosorbent assay (ELISA)-based quantitation assays have not been performed due to the lack of suitable antibody. Although many diagnostic platforms are based on the ELISA principle, the lack of a specific anti-GTA-446 antibody represents a limiting factor for GTA-446 detection and quantification. Production of a specific antibody requires sufficient quantities of pure compound antigen, which has been limited by the unavailability of synthetic GTA-446.

Alternative, additional, and/or improved sources of GTA-446 and/or derivatives thereof, and/or methods for quantification thereof in a sample, are desirable.

### SUMMARY OF INVENTION

It is an object of the invention to provide compounds having the structure of formula I, or salts, esters, prodrugs, or labelled derivatives thereof, or compounds related thereto, which may be of use in assays for quantifying GTA levels, such as GTA-446 levels, in a sample and/or in assays for the diagnosis of colorectal cancer in a subject.

In certain embodiments, there is provided herein a compound having the structure of formula I or formula III:

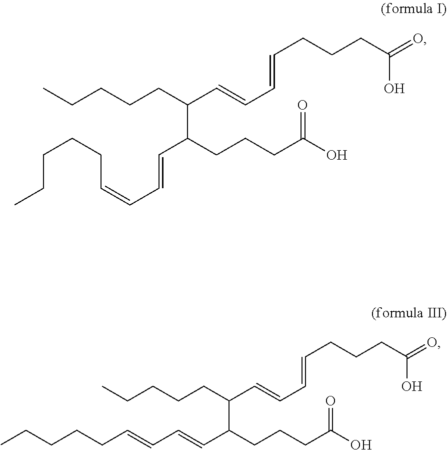 [SMILES: CCCCC/C=C\C=C\C(CCCC(=O)O)C(/C=C/C=C/CCCC(=O)O)CCCCC ; CCCCC/C=C/C=C/C(CCCC(=O)O)C(/C=C/C=C/CCCC(=O)O)CCCCC]

or a salt, ester, prodrug, or labelled derivative thereof.

In a further embodiment, the compound may an isolated compound.

In another embodiment of the above compound or compounds, the compound may be a synthetically prepared compound, an analytical standard compound, or both.

In still another embodiment, there is provided herein an isotopically labelled compound, the isotopically labelled compound comprising one or more isotopic labels incorporated within the structure of formula I or formula III:

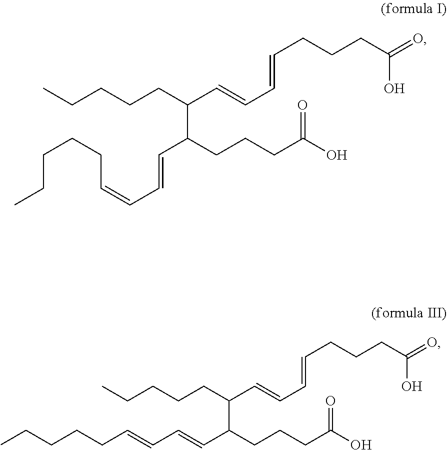 [SMILES: CCCCC/C=C\C=C\C(CCCC(=O)O)C(/C=C/C=C/CCCC(=O)O)CCCCC ; CCCCC/C=C/C=C/C(CCCC(=O)O)C(/C=C/C=C/CCCC(=O)O)CCCCC]

or a salt, ester, or prodrug thereof.

In a further embodiment of the isotopically labelled compound above, the one or more isotopic labels may be stable isotope labels, radioisotope labels, or a combination thereof.

In still a further embodiment of the isotopically labelled compound or compounds above, the one or more isotopic labels may be selected from the group consisting of deuterium (²H) and ¹³C.

In still a further embodiment of the isotopically labelled compound or compounds above, the one or more isotopic labels may be selected from the group consisting of tritium (³H) and ¹⁴C.

In still another embodiment, the isotopically labelled compound may be:

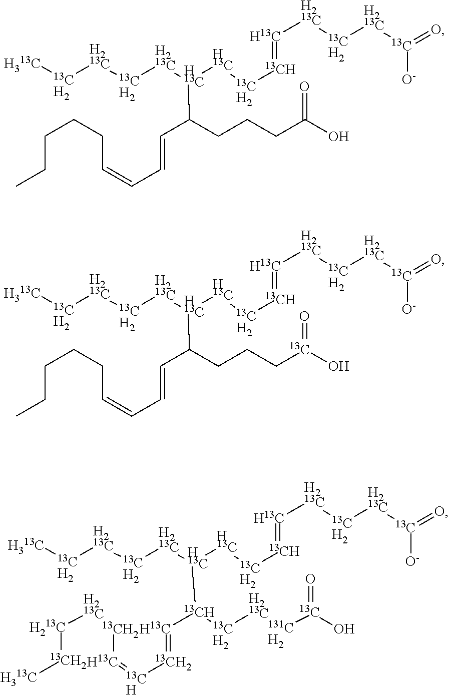

or a derivative thereof in which all carbon-carbon double bonds are in trans configuration,
or a salt, ester, or prodrug thereof.

In yet another embodiment, the isotopically labelled compound or compounds above may be an analytical standard compound.

In another embodiment of the compound or compounds above, the compound may be a compound of:

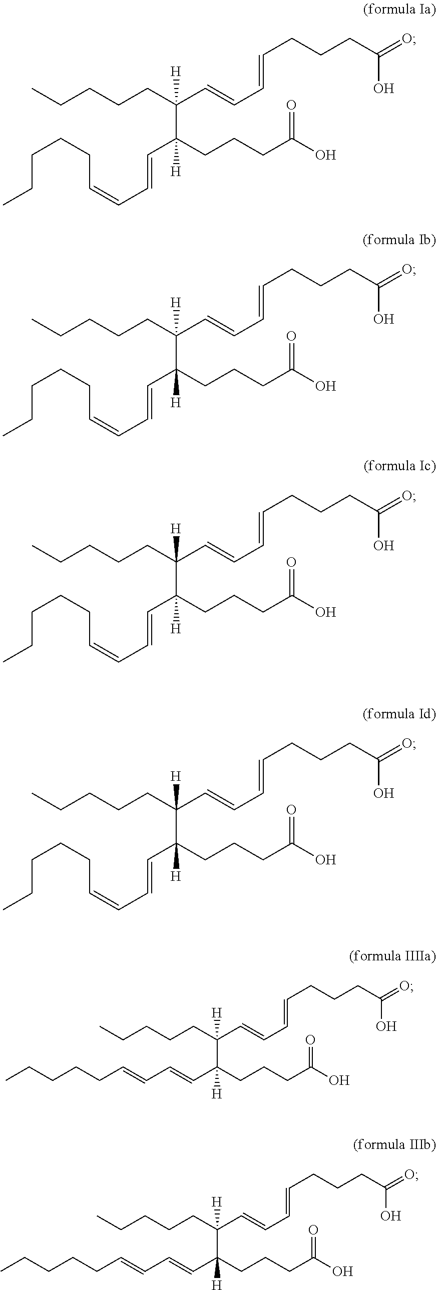 [SMILES: CCCCC/C=C\C=C\[C@H](CCCC(=O)O)[C@H](/C=C/C=C/CCCC(=O)O)CCCCC ; CCCCC/C=C\C=C\[C@@H](CCCC(=O)O)[C@H](/C=C/C=C/CCCC(=O)O)CCCCC ; CCCCC/C=C\C=C\[C@H](CCCC(=O)O)[C@@H](/C=C/C=C/CCCC(=O)O)CCCCC ; CCCCC/C=C\C=C\[C@@H](CCCC(=O)O)[C@@H](/C=C/C=C/CCCC(=O)O)CCCCC ; CCCCC/C=C/C=C/[C@H](CCCC(=O)O)[C@H](/C=C/C=C/CCCC(=O)O)CCCCC ; CCCCC/C=C/C=C/[C@@H](CCCC(=O)O)[C@H](/C=C/C=C/CCCC(=O)O)CCCCC]

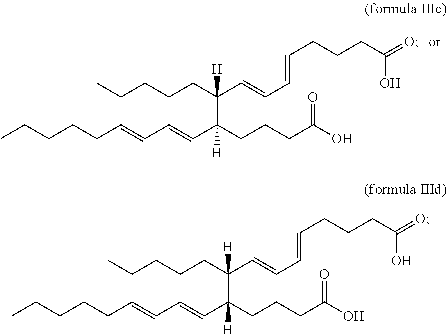 [SMILES: CCCCC/C=C/C=C/[C@H](CCCC(=O)O)[C@@H](/C=C/C=C/CCCC(=O)O)CCCCC ; CCCCC/C=C/C=C/[C@@H](CCCC(=O)O)[C@@H](/C=C/C=C/CCCC(=O)O)CCCCC]

or any combination thereof;
   or a salt, ester, prodrug, or labelled derivative thereof.

In another embodiment, there is provided herein a metabolic tracer composition comprising isotopically labelled compound or compounds as described above.

In still another embodiment, there is provided herein a composition comprising any of the compound or compounds above, and an excipient, carrier, or diluent.

In yet another embodiment, there is provided herein an in vitro or in vivo diagnostic agent comprising isotopically labelled compound or compounds as described above.

In another embodiment, there is provided herein a composition comprising any of the compound or compounds above, and an excipient, carrier, or diluent.

In yet another embodiment, there is provided herein a method for determining a level of a gastric tract acid (GTA) in a sample, said method comprising:
measuring a GTA detection signal from the sample, the GTA detection signal representative of the GTA level in the sample; and
quantifying the level of the GTA in the sample by comparing the measured GTA detection signal with a calibration reference.

In a further embodiment of the above method, the GTA may be

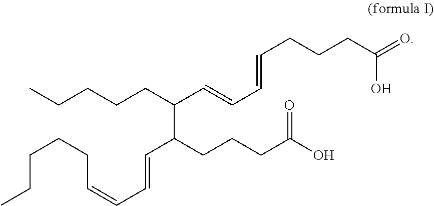 [SMILES: CCCCC/C=C\C=C\C(CCCC(=O)O)C(/C=C/C=C/CCCC(=O)O)CCCCC]

In yet another embodiment of the above method or methods, the GTA detection signal may be measured by mass spectrometry.

In still another embodiment of the above method or methods, the calibration reference may comprise a standard curve prepared using known quantities of compound or compounds as defined above.

In yet another embodiment of the above method or methods, the calibration reference may be obtained by:
spiking the sample with a known quantity of isotopically labelled compound or compounds as defined above; and
measuring an internal standard signal from the sample, the internal standard signal being representative of the known quantity of the isotopically labelled compound spiked into the sample.

In a further embodiment of the above method or methods, the internal standard signal may be measured by mass spectrometry.

In yet another embodiment of the above method or methods, the method may further comprise a step of determining a ratio of the GTA level in the sample, as represented by the measured GTA detection signal, to the known quantity of isotopically labelled compound spiked into the sample, as represented by the internal standard signal.

In yet another embodiment of the above method or methods, the calibration reference may comprise an isotope dilution curve (IDC) generated from a series of mixtures of varying GTA/isotopically labelled compound ratios and concentrations, to which said ratio is compared.

In still another embodiment of the above method or methods, the IDC may be generated from a series of mixtures in which GTA content is varied over a fixed amount of isotopically labelled compound.

In another embodiment of the above method or methods, the fixed amount of the isotopically labelled compound may be substantially the same as the known quantity of the isotopically labelled compound which is spiked into the sample.

In another embodiment, there is provided herein a use of compound or compounds as defined above for determining a level of a gastric tract acid (GTA) in a sample. In a further embodiment, the GTA may be:

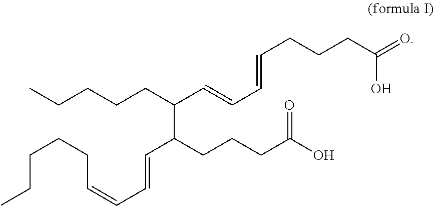 [SMILES: CCCCC/C=C\C=C\C(CCCC(=O)O)C(/C=C/C=C/CCCC(=O)O)CCCCC]

In yet another embodiment, there is provided herein a use of compound or compounds as defined above for generating a calibration reference for use in determining a level of a gastric tract acid (GTA) in a sample. In a further embodiment, the GTA may be:

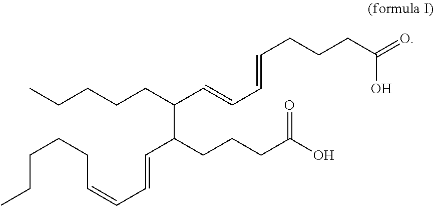 [SMILES: CCCCC/C=C\C=C\C(CCCC(=O)O)C(/C=C/C=C/CCCC(=O)O)CCCCC]

In another embodiment, there is provided herein a use of compound or compounds as defined above as an internal standard for use in determining a level of a gastric tract acid (GTA) in a sample. In a further embodiment, the GTA may be:

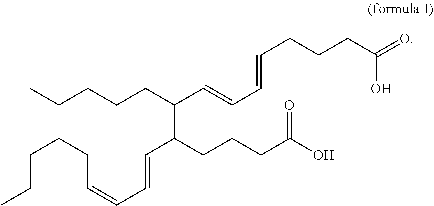 [SMILES: CCCCC/C=C\C=C\C(CCCC(=O)O)C(/C=C/C=C/CCCC(=O)O)CCCCC]

In another embodiment, there is provided herein a diagnostic method for identifying a subject as having, or being at risk of developing, colorectal cancer, said method comprising:
determining a level of a gastric tract acid (GTA) in a sample obtained from the subject by
   measuring a GTA detection signal from the sample, the GTA detection signal representative of the GTA level in the sample; and
   quantifying the level of the GTA in the sample by comparing the measured GTA detection signal with a calibration reference, and
identifying the subject as having, or being at risk of developing, colorectal cancer when the determined level of the GTA in the sample is reduced in comparison to a healthy control group,

wherein the GTA is:

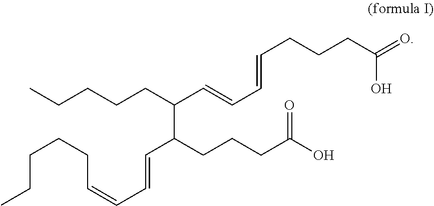 [SMILES: CCCCC/C=C\C=C\C(CCCC(=O)O)C(/C=C/C=C/CCCC(=O)O)CCCCC]

In a further embodiment, the GTA detection signal may be measured by mass spectrometry.

In yet another embodiment of the above method or methods, the calibration reference may comprise a standard curve prepared using known quantities of compound or compounds as defined above.

In another embodiment of the above method or methods, the step of determining the level of the GTA in the sample obtained from the subject may comprise:
spiking the sample with a known quantity of isotopically labelled compound or compounds as defined above; and
measuring an internal standard signal from the sample, the internal standard signal being representative of the known quantity of the isotopically labelled compound spiked into the sample.

In a further embodiment, the internal standard signal may be measured by mass spectrometry.

In yet another embodiment of the above method or methods, the method may further comprise a step of determining a ratio of the GTA level in the sample, as represented by the measured GTA detection signal, to the known quantity of isotopically labelled compound spiked into the sample, as represented by the internal standard signal.

In still another embodiment, of the above method or methods, the calibration reference may comprise an isotope dilution curve (IDC) generated from a series of mixtures of varying GTA/isotopically labelled compound ratios and concentrations, to which said ratio is compared.

In another embodiment, the IDC may be generated from a series of mixtures in which GTA content is varied over a fixed amount of isotopically labelled compound.

In yet another embodiment of the above method or methods, the fixed amount of the isotopically labelled compound may be substantially the same as the known quantity of the isotopically labelled compound which is spiked into the sample.

In another embodiment, there is provided herein a use of compound or compounds as defined above in a diagnostic method for identifying a subject as having, or being at risk of developing, a colorectal cancer associated with altered levels of a gastric tract acid (GTA) which is:

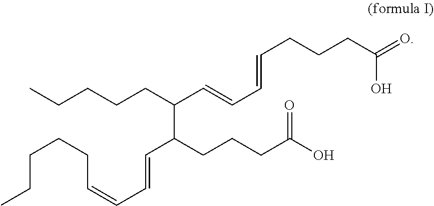 [SMILES: CCCCC/C=C\C=C\C(CCCC(=O)O)C(/C=C/C=C/CCCC(=O)O)CCCCC]

In still another embodiment, there is provided herein a use of compound or compounds as defined above for generating a calibration reference for use in a diagnostic method for identifying a subject as having, or being at risk of developing, a colorectal cancer associated with altered levels of a gastric tract acid (GTA) which is:

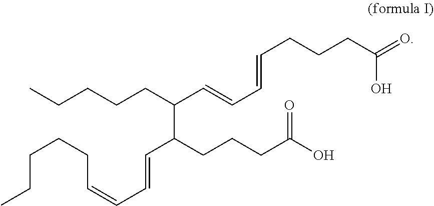 [SMILES: CCCCC/C=C\C=C\C(CCCC(=O)O)C(/C=C/C=C/CCCC(=O)O)CCCCC]

In another embodiment, there is provided herein a use of the compound or compounds defined above as an internal standard for use in a diagnostic method for identifying a subject as having, or being at risk of developing, a colorectal cancer associated with altered levels of a gastric tract acid (GTA) which is:

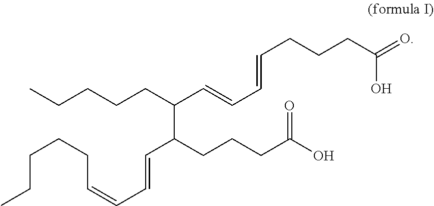 [SMILES: CCCCC/C=C\C=C\C(CCCC(=O)O)C(/C=C/C=C/CCCC(=O)O)CCCCC]

In another embodiment, there is provided herein an antibody, or antigen-binding fragment thereof, which specifically binds a compound of formula I:

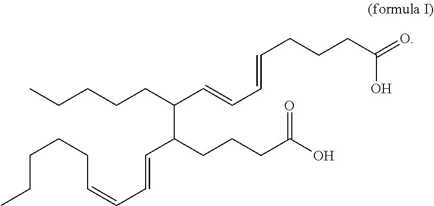 [SMILES: CCCCC/C=C\C=C\C(CCCC(=O)O)C(/C=C/C=C/CCCC(=O)O)CCCCC]

In another embodiment, the antibody may be a monoclonal or a polyclonal antibody.

In another embodiment, there is provided herein a use of the compound or compounds defined above as an antigen for preparing an antibody which specifically binds to an antigenic epitope of the compound.

In yet another embodiment, there is provided herein a use of the antibody or antibodies defined above for detecting or quantifying a level of a gastric tract acid (GTA) in a sample by immunoassay, wherein the GTA is:

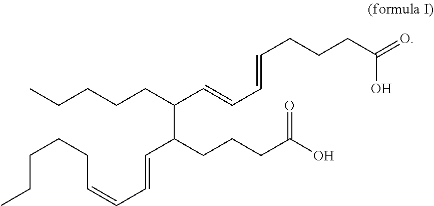 [SMILES: CCCCC/C=C\C=C\C(CCCC(=O)O)C(/C=C/C=C/CCCC(=O)O)CCCCC]

In still another embodiment, there is provided herein a use of the antibody or antibodies defined above in a diagnostic method for identifying a subject as having, or being at risk of developing, a colorectal cancer associated with altered levels of a gastric tract acid (GTA) which is:

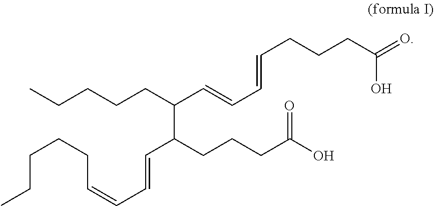 [SMILES: CCCCC/C=C\C=C\C(CCCC(=O)O)C(/C=C/C=C/CCCC(=O)O)CCCCC]

In another embodiment, there is provided herein a method for determining a level of a gastric tract acid (GTA) in a sample, said method comprising:
measuring a level of the GTA in the sample using an immunoassay employing an antibody, or antigen-binding fragment thereof, which specifically binds to the GTA;
wherein the GTA is:

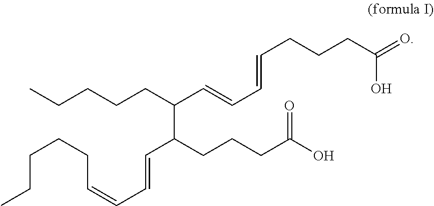 [SMILES: CCCCC/C=C\C=C\C(CCCC(=O)O)C(/C=C/C=C/CCCC(=O)O)CCCCC]

In a further embodiment of the above method, the immunoassay may comprise an enzyme-linked immunosorbent assay (ELISA).

In another embodiment of the above method or methods, the method may further comprise a step of using a control sample comprising compound or compounds as defined above as a positive control in the immunoassay.

In yet another embodiment of the above method or methods, the method may further comprise a step of using a standard curve to extrapolate the level of the GTA in the sample, the standard curve having been generated using a plurality of known quantities of a compound or compounds as defined above.

In another embodiment, there is provided herein a diagnostic method for identifying a subject as having, or being at risk of developing, colorectal cancer, said method comprising:
determining a level of a gastric tract acid (GTA) in a sample obtained from the subject by
   measuring a level of the GTA in the sample using an immunoassay employing an antibody, or antigen-binding fragment thereof, which specifically binds to the GTA; and
identifying the subject as having, or being at risk of developing, colorectal cancer when the determined level of the GTA in the sample is reduced in comparison to a healthy control group,

wherein the GTA is:

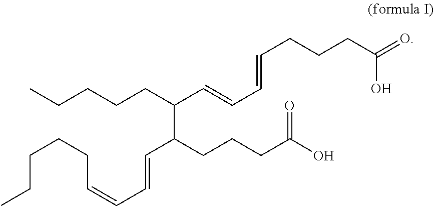 [SMILES: CCCCC/C=C\C=C\C(CCCC(=O)O)C(/C=C/C=C/CCCC(=O)O)CCCCC]

In a further embodiment, the immunoassay may comprise an enzyme-linked immunosorbent assay (ELISA).

In another embodiment of the above method or methods, the method may further comprise a step of using a control sample comprising a compound or compounds as defined above as a positive control in the immunoassay.

In still another embodiment of the above method or methods, the step of determining the level of the GTA in the sample may further comprise using a standard curve to extrapolate the level of the GTA in the sample, the standard curve having been generated using a plurality of known quantities of a compound or compounds as defined above.

In another embodiment, there is provided herein a kit for quantifying a level of a gastric tract acid (GTA) in a sample, the kit comprising at least one of:
a compound or compounds as defined above;
a metabolic tracer as defined above;
a composition or compositions as defined above;
a diagnostic agent as defined above; and an antibody or antibodies as defined above;
and, optionally, further comprising a set of instructions for performing a method or methods as defined above.

In another embodiment, there is provided herein a diagnostic kit for identifying a subject as having, or being at risk of developing, colorectal cancer, the kit comprising at least one of: a compound or compounds as defined above;
a metabolic tracer as defined above;
a composition or compositions as defined above;
a diagnostic agent as defined above; and an antibody or antibodies as defined above;
and, optionally, further comprising a set of instructions for performing a method or methods as defined above.

In another embodiment, there is provided herein a compound having the formula:

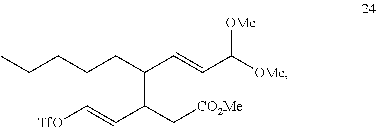 [SMILES: CCCCCC(/C=C/C(OC)OC)C(/C=C/OS(=O)(=O)C(F)(F)F)CC(=O)OC]

or a labelled derivative thereof.

In still another embodiment, there is provided herein a use of the above compound in the synthesis of a compound having the formula:

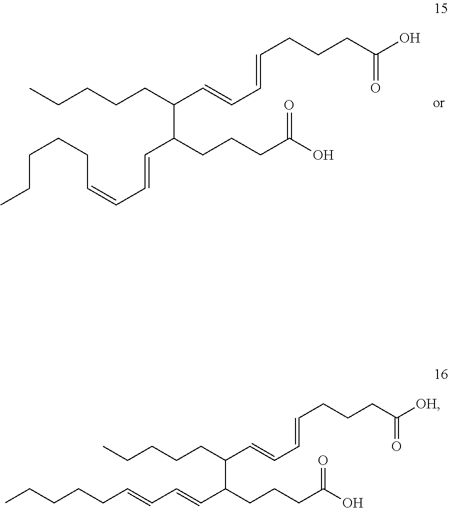 [SMILES: CCCCC/C=C\C=C\C(CCCC(=O)O)C(/C=C/C=C/CCCC(=O)O)CCCCC ; CCCCC/C=C/C=C/C(CCCC(=O)O)C(/C=C/C=C/CCCC(=O)O)CCCCC]

a salt, ester, prodrug, or labelled derivative thereof.

In another embodiment, there is provided herein a method for synthesizing a compound having formula (I):

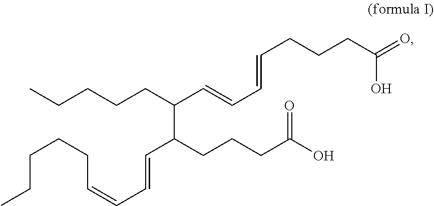 [SMILES: CCCCC/C=C\C=C\C(CCCC(=O)O)C(/C=C/C=C/CCCC(=O)O)CCCCC]

or an isotopically labelled derivative thereof,
said method comprising:
providing a compound having the formula

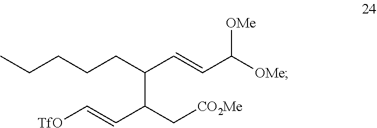 [SMILES: CCCCCC(/C=C/C(OC)OC)C(/C=C/OS(=O)(=O)C(F)(F)F)CC(=O)OC]

performing a Sonagashira coupling of the compound with 1-heptyne;
performing a reduction with Lindlar's catalyst;
performing a methyl ester reduction;
performing a reaction with methanesulfonyl chloride;
performing a mesylate displacement with dimethyl malonate;
performing an acid treatment for simultaneous acetal cleavage, ester hydrolysis, and decarboxylation; and
performing a Wittig reaction to yield the compound of formula I, or an isotopically labelled derivative thereof,
wherein the compound of formula 24, or at least one reactant in the method, comprises at least one isotopically labelled atom which is incorporated into the resulting compound of formula I when an isotopically labelled derivative of formula I is synthesized.

In a further embodiment, the Wittig reaction may comprise reaction with (triphenylphosphoranylidene) acetaldehyde or (4-carboxybutyl)triphenylphosphonium bromide.

In yet another embodiment, there is provided herein a compound having formula D:

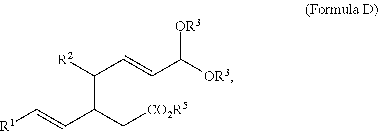

wherein:
R¹ is —Sn(R¹⁰)₃, —OTf, —Cl, —Br, —I, —B(OH)₂ or

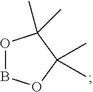

R² is optionally substituted saturated or unsaturated C₁-C₂₀ alkyl, saturated or unsaturated C₂-C₂₀ alkenyl, or saturated or unsaturated C₂-C₂₀ alkynyl;
each R³ is, independently, optionally substituted C₁-C₆ alkyl, or the R³ groups together form an optionally substituted ethylene or propylene group bridging the attached oxygen atoms to form a five- or six-membered ring;
R⁵ is optionally substituted C₁-C₆ alkyl; and
R¹⁰ is optionally substituted C₁-C₆ alkyl,

or a labelled derivative thereof.

In still another embodiment, there is provided herein a use of the compound of formula D in the synthesis of a gastric tract acid (GTA), or a derivative thereof. In certain embodiments, the GTA or the derivative thereof may be a compound of formula N or S:

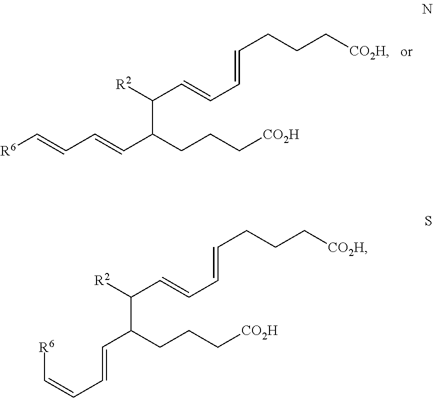

or a salt, ester, prodrug, or labelled derivative thereof,

wherein
R² is optionally substituted saturated or unsaturated C₁-C₂₀ alkyl, saturated or unsaturated C₂-C₂₀ alkenyl, or saturated or unsaturated C₂-C₂₀ alkynyl; and
R⁶ is optionally substituted saturated or unsaturated C₁-C₂₀ alkyl, saturated or unsaturated C₂-C₂₀ alkenyl, or saturated or unsaturated C₂-C₂₀ alkynyl.

In another embodiment, there is provided herein a method for synthesizing a compound of formula N or S as defined above, or an isotopically labelled derivative thereof, said method comprising:
providing a compound of formula D as defined above;
performing a coupling reaction and, optionally, a reduction, to replace the R¹ group with an optionally substituted saturated or unsaturated alkyl, saturated or unsaturated alkenyl, or saturated or unsaturated alkynyl;
converting the R⁵-containing ester to a hydroxyl group;
converting the hydroxyl group to a leaving group;
displacing the leaving group with a dialkyl malonate;
performing acetal hydrolysis, ester hydrolysis, and decarboxylation, forming an aldehyde; and
performing a coupling reaction at the aldehyde to yield the compound of formula N or S, or an isotopically labelled derivative thereof,

wherein the compound of formula D, or at least one reactant in the method, comprises at least one isotopically labelled atom which is incorporated into the resulting compound of formula N or S when an isotopically labelled derivative of formula N or S is synthesized.

### BRIEF DESCRIPTION OF DRAWINGS

FIG. 1 shows a typical total ion flow injection chromatogram (TIC) of a regular serum organic extract in water saturated ethyl acetate in negative APCI before any method development. This Figure shows how low GTA-446 in a comprehensive serum matrix;

FIG. 2 shows a full scan column chromatogram (RP-18) of a regular serum organic extracts in water saturated ethyl acetate showing GTA elution window of 16-18 minutes using methanol water gradient;

FIG. 3 shows a comparison of CRC 446 (MRM 445/383) levels in the organic phase between monophasic extractions (MeOH-1-MeOH-3) versus biphasic extractions (N-1-N3). Monophasic extraction was implemented based on this data;

FIG. 4 shows a total ion current flow injection chromatogram of the upper organic phase using the monophasic extraction followed by phase separation. GTA 446 is observed as m/z 445.3;

FIG. 5 shows a full scan flow injection chromatogram of normal phase flash column chromatography fraction 3 (F3) in NAPCI showing enrichment of GTAs compared to the crude matrix;

FIG. 6 shows a full scan flow injection chromatogram of reverse phase flash column chromatography fraction 5 (F5) in NAPCI showing further purification of GTA 446;

FIG. 7 shows a full scan flow injection chromatogram of reverse phase flash column chromatography fraction 6 (F6) in NAPCI showing further purification of GTA 446;

FIG. 8 shows a full scan chromatogram of GTA-446 rich sample in NAPCI from prep HPLC-RP separation;

FIG. 9 shows a full scan chromatogram from prep HPLC-NP separation in NAPCI showing purified GTA 446;

FIG. 10 shows tandem MS spectra of isolated GTA-446 in NAPCI CE=35 v showing similar fragmentation fingerprint to its endogenous version found in comprehensive serum matrix;

FIG. 11 shows a ¹H-NMR of isolated GTA-446 biomarker_2 (in CDCl₃) showing 8 methylene protons (δ 5.5 to 6.2 pm), two terminal methyl groups (—CH₂CH₃, δ 0.84 and 0.89 pm, each 3H, t), and a broad peak at δ 12.0 ppm from two —COOH groups as key functional groups;

FIG. 12 shows a ¹³C-NMR of isolated GTA-446 biomarker 2 (in CDCl₃) showing 8 methylene carbons (δ 126 to 136 pm), two terminal methyl groups (—CH₂CH₃, δ 14.0 and 14.1 pm), and two carbonyl carbons at δ 181.2, 181.4 ppm from two —COOH groups, two methylene carbons at 45.9, 47.0 ppm as unique structural entities;

FIG. 13 shows a ¹H-¹H COSY analysis of GTA-446 biomarker_2 (in CDCl₃) showing 2D proton correlations. The E,E and E,Z configuration was deduced from their coupling constants;

FIG. 14 shows a direct ¹H-¹³C (HMQC) analysis of isolated GTA-446 biomarker_2 (in CDCl₃) showing direct 2D ¹³C-¹H correlations;

FIG. 15 shows long range ¹H-¹³C (HMBC) analysis of isolated GTA-446 biomarker 2 (in CDCl₃) showing vicinal 2D ¹³C-_¹H correlations;

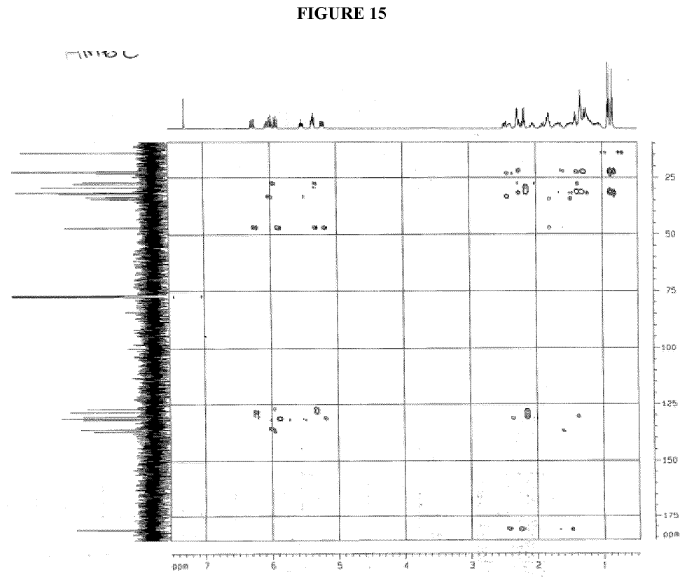

FIG. 16 shows proposed ¹³C stable isotope forms for GTA-446 and their tandem MS fragments predicted based on the naturally occurring form;

FIG. 17 shows a serum interference check for proposed ¹³C stable isotopes of GTA-446 using 25 individual human serum samples. This shows that both predicted structures A and B has the lowest serum interference, and may be candidates to for use as internal standards in an isotopic dilution method to measure endogenous levels of GTA-446; and

FIG. 18 shows an example of a process embodiment outline for commercial manufacture of GTA-446 for use as a commercial standard in a CRC blood test.

### DETAILED DESCRIPTION

Described herein are gastric tract acid (GTA)-based compounds having the structure of formula I, as well as salts, esters, prodrugs, or labelled derivatives thereof, and other compounds relating thereto. Such GTA compounds may be used for determining GTA levels of a sample, for diagnosing a subject as having or being at risk of developing colorectal cancer, or for raising antibodies. Antibodies, or fragments thereof, which specifically bind to the GTA of formula I (or related compounds) are described, as well as uses of such antibodies or fragments thereof for determining GTA levels in a sample, or for diagnosing a subject as having or being at risk of developing colorectal cancer. Kits comprising such GTA compounds and/or antibodies are also provided.

It will be appreciated that embodiments and examples are provided for illustrative purposes intended for those skilled in the art, and are not meant to be limiting in any way.

In certain embodiments, there is provided herein a gastric tract acid (GTA) compound having the structure of formula I:

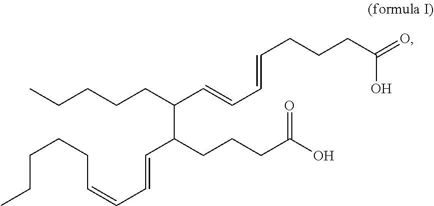 [SMILES: CCCCC/C=C\C=C\C(CCCC(=O)O)C(/C=C/C=C/CCCC(=O)O)CCCCC]

or a salt, ester, prodrug, or labelled derivative thereof.

In certain embodiments, the GTA compound may include a compound having the structure of formula II:

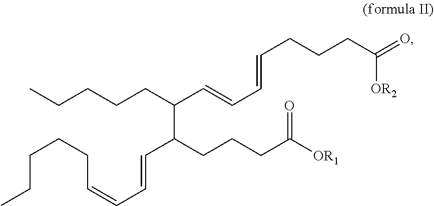

wherein R₁ and R₂ are each, independently, hydrogen; a counter ion; a linear or branched substituted or unsubstituted alkane, alkene, or alkyne; a substituted or unsubstituted cycloalkane, cycloalkene, or cycloalkyne; a substituted or unsubstituted aromatic group; a promoiety of a prodrug; a fluorophore; or a protecting group; or wherein —OR₁ and/or —OR₂ may be each, independently, replaced by a biocleavable functional group which may be processed in vitro or in vivo to form a —COOH group or salt thereof.

As will be understood, in certain embodiments, compounds of formula I and/or II may be provided as a mixture of stereoisomers (which may be generally racemic, or may be at least partially enriched in one or more of the stereoisomers), or may be provided in substantially stereoisomerically pure form. Compounds of formulas I and II include two chiral carbons, and may be configured as R/R, R/S, S/R, and S/S diastereomers. Thus, in certain embodiments, there is provided herein a GTA compound having a structure as follows:

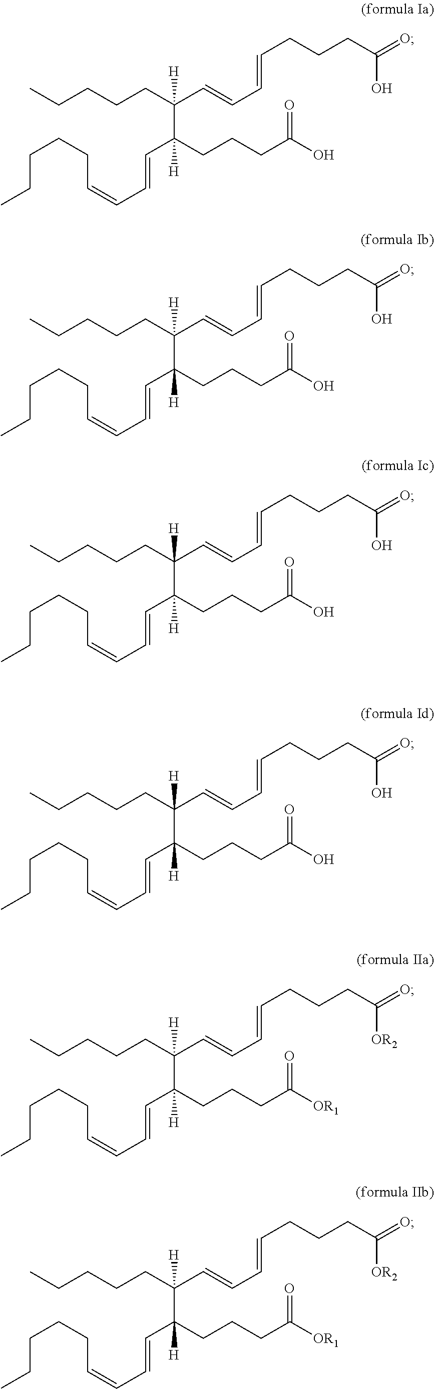

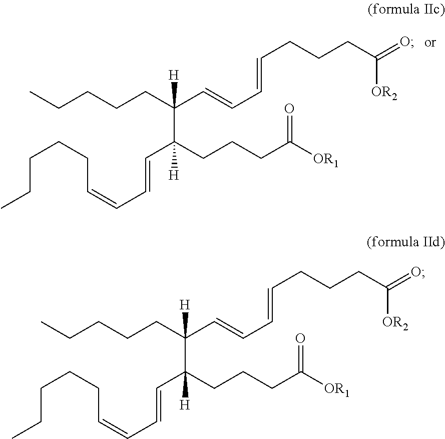

or any mixture thereof.

In certain embodiments, by way of example, a counter ion may include any suitable counter ion to counterbalance a negative charge on the —COO— group, thereby forming a salt. Non-limiting examples may include, for example, sodium, potassium, lithium, ammonium, or an alkylammonium.

Examples of a promoiety of a prodrug may include, for example, a methyl, ethyl, propyl, or isopropyl group, a hydrophobic group, a membrane transport peptide or signal, a membrane-traversing moiety, a cell-targeting moiety, or another suitable group which is biocleavable in vitro or in vivo.

Examples of a fluorophore may include, for example, a fluorescein, cyanine, GFP, YFP, RFP, or other such fluorophore, dye, or commercially available label.

Examples of a protecting group may include methyl esters, benzyl esters, tert-butyl esters, oxazoline, or silyl esters, for example. The person of skill in the art will be aware of a variety of protecting groups, many of which are described in Greene's Protective Groups in Organic Synthesis, Fourth Ed., ISBN: 9780471697541 (2007; John Wiley & Sons, Inc.), which is herein incorporated by reference in its entirety.

Examples of a biocleavable functional group which may be processed in vivo or in vitro to form a —COOH group or salt thereof may include any suitable carbonate, ester, amide, or carbamate group, for example.

In certain further embodiments, the GTA compound may include an isotopically labelled derivative of any of the compounds described above. In certain embodiments, the isotopically labelled derivative may include one, or more than one, isotopic labels integrated therein. In certain embodiments, the one or more isotopic labels may include stable isotope labels, radioisotope labels, or a combination thereof. The isotopic label(s) may, for example, comprise deuterium (²H) or ¹³C, or both. In certain embodiments, the isotopic label(s) may, for example, comprise tritium (³H), ¹⁴C, or both. In certain embodiments the isotopic label may comprise at least one deuterium or tritium label covalently bound to a carbon atom, for example, at a saturated carbon within the GTA structure. The skilled person having regard to the teachings herein will be aware of a variety of labels (isotopic, radioisotopic, fluorescent, or other) which may be incorporated with, or linked to, the compounds described above in order to suit a particular application.

In certain embodiments, there is also provided herein compounds related to the gastric tract acid (GTA) compounds described above, which may share similar properties. In certain embodiments, there is provided herein a compound having the structure of formula III:

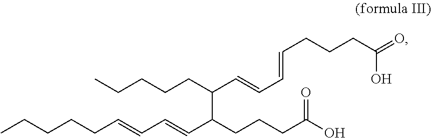 [SMILES: CCCCC/C=C/C=C/C(CCCC(=O)O)C(/C=C/C=C/CCCC(=O)O)CCCCC]

or a salt, ester, prodrug, or labelled derivative thereof.

In another embodiment, there is provided herein a compound of formula IV:

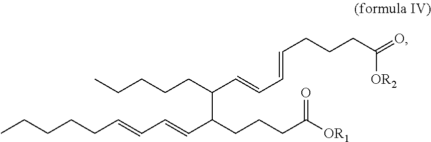

wherein R₁ and R₂ are as defined above with reference to formula II.

As will be understood, in certain embodiments, compounds of formula III and/or IV may be provided as a mixture of stereoisomers (which may be generally racemic, or may be at least partially enriched in one or more of the stereoisomers), or may be provided in substantially stereoisomerically pure form. Compounds of formulas III and IV include two chiral carbons, and may be configured as R/R, R/S, S/R, and S/S diastereomers. Thus, in certain embodiments, there is provided herein a compound having a structure as follows:

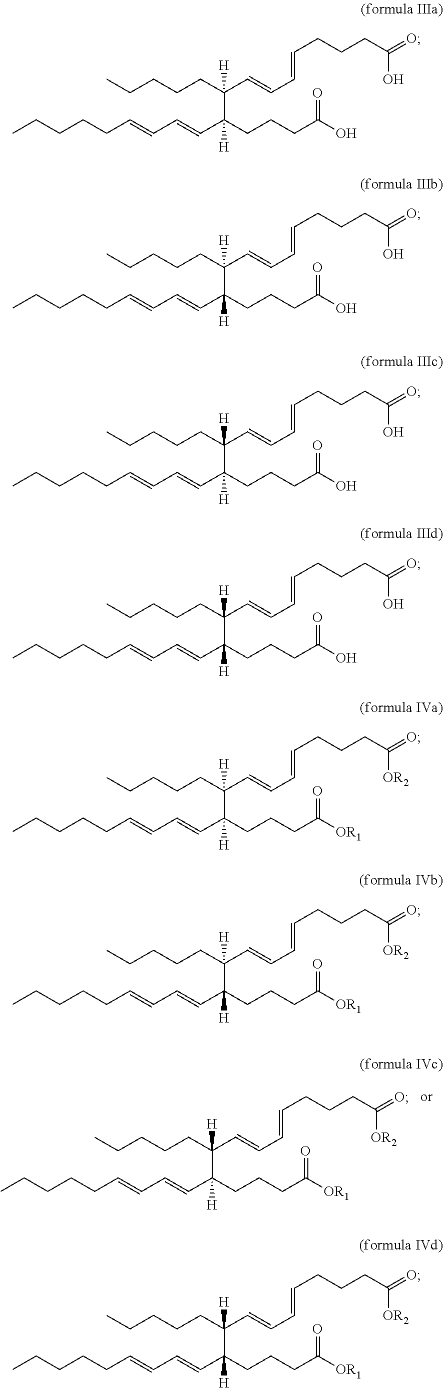

or any mixture thereof.

In certain further embodiments, the compound of formula III or IV may include an isotopically labelled derivative of any of the compounds described above. In certain embodiments, the isotopically labelled derivative may include one, or more than one, isotopic labels integrated therein. In certain embodiments, the one or more isotopic labels may include stable isotope labels, radioisotope labels, or a combination thereof. The isotopic label(s) may, for example, comprise deuterium (²H) or ¹³C, or both. In certain embodiments, the isotopic label(s) may, for example, comprise tritium (³H), ¹⁴C, or both. In certain embodiments the isotopic label may comprise at least one deuterium or tritium label covalently bound to a carbon atom, for example, at a saturated carbon within the structure. The skilled person having regard to the teachings herein will be aware of a variety of labels (isotopic, radioisotopic, fluorescent, or other) which may be incorporated with, or linked to, the compounds described above in order to suit a particular application.

Further embodiments of such compounds, and contemplated uses thereof, are described in further detail below.

Gastric Tract Acids and GTA-446

Provided herein are polyunsaturated 28-carbon dicarboxylic fatty acids, including the gastric tract acid GTA-446, and derivatives thereof. Such compounds may, for example, be for use in improving accuracy and/or specificity of assays which quantify the levels of said 28-carbon fatty acid, or another GTA, in serum and/or other biological matrices.

In certain embodiments, compounds as described herein may be used, for example, as reference standards or calibration references in the quantification of related molecules including, for example, the gastric tract acid GTA-446 or another such GTA in a sample such as, for example, a biospecimen which may, in certain embodiments, be a human serum sample or other such sample.

The gastric tract acid GTA-446 has not been previously fully characterized, has not been synthetically prepared, and labelled derivatives have not been generated. Furthermore, until now, GTA-446 (C₂₈H₄₆O₄) was thought to be a single long-chain fatty acid containing four unsaturations, a single carboxylic acid moiety, and two hydroxy moieties.

However, the experimental studies detailed herein have now revealed that GTA-446 is actually a dicarboxylic fatty acid dimer comprising a conjugation of two 14-carbon unsaturated fatty acids (see formula I below). Specifically, GTA-446 is bridged at the 9- and 5′-position, and comprises two terminal carboxylic acid groups. Based on these results, it appears likely that the GTA family in general may be comprised of long chain (C₁₄-C₂₂) polyunsatured fatty acid dimers bridged between their alkyl backbones. To the best of our knowledge, this work represents the first instance where molecules having this structure have been reported, particularly in human serum.

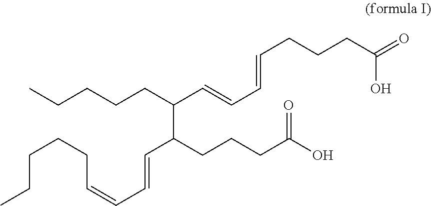 [SMILES: CCCCC/C=C\C=C\C(CCCC(=O)O)C(/C=C/C=C/CCCC(=O)O)CCCCC]

Molecular Structure of GTA-446 as Derived from NMR (¹H, ¹³C, COSY, HMBC and HMQC) and Mass Spectroscopy (FTICR and MS/MS). Chemical Formula C₂₈H₄₆O₄. Exact Mass: 446.34

In certain embodiments, there is therefore provided herein a purified or isolated GTA-446 compound or composition, which may, for example, be of use as an analytical standard. Also provided herein are isolation processes for obtaining purified compounds of formula I from a GTA-446-rich starting material such as human serum, involving a multi-step process which may include steps of precipitation, phase separation, FCC and/or HPLC separation of the target to achieve high purity.

In another embodiment, there are provided herein methods to produce a labelled isoform of GTA-446 (and other compounds related thereto) by incorporating one or more labels into the purified GTA-446 isoform. By way of example, such labels might be incorporated by methods comprising a step of deuterium exchange, or another suitable modification step selected from various forms of derivatization which may include Diels-Alder derivatization of the conjugated system using suitable commercially available reagents (for example, PTAD, 4-Phenyl-1,2,4-triazoline-3,5-dione), as well as various carboxylic acid derivatives. In certain embodiments, where the labelled isoform of GTA-446 is for use in a mass spectrometry application, the labelled isoform of GTA-446 may be designed such that the mass of the parent-daughter ions are not in common with the unlabeled GTA-446 analyte (or other analyte) being identified or quantified by the MS analysis. Examples of rationally designed isotopically labelled compounds are described in further detail below.

In certain embodiments, there is provided herein a compound having the structure of formula I:

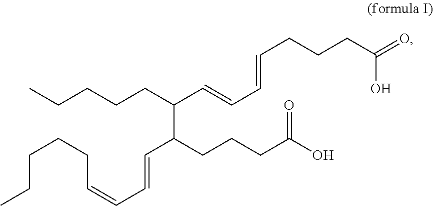 [SMILES: CCCCC/C=C\C=C\C(CCCC(=O)O)C(/C=C/C=C/CCCC(=O)O)CCCCC]

or a salt, ester, prodrug, or labelled derivative thereof.

Also provided are related compounds, such as those of formulas II, III, and IV described above.

In certain embodiments, the compound may be an isolated or purified compound. The compound may, for example, be a synthetically prepared compound. The compound may, in certain embodiments, be prepared as an analytical standard compound for use in an assay.

Also provided herein are compositions comprising such compound(s), and an acceptable excipient, carrier, or diluent.

Isolation from Human Serum

Examples of processes for isolating GTA-446 from human serum are described in detail in Examples 1 and 2 below. Processes may, in certain embodiments, involve the evaluation of lots of commercially available serum to identify those with high GTA-446 concentration, followed by procurement of large (50 L) quantities of the chosen lot. In certain embodiments, the test lots of serum may be extracted between water and ethyl acetate buffered with formic acid (thereby precipitating out protein). In the studies of Example 1, it was observed that GTA-446 eluted with a pool of nine other similar fatty acids between 16.3-17.6 minutes under the selected method (see below). Selection of the serum source may be decided based on how clean and/or enriched this starting extraction for GTA-446 is, which may be useful in facilitating scale-up extractions since other contaminants may reduce the efficiency of purification.

The experiments described in further detail hereinbelow have identified methods for isolating GTA-446 for a biological fluid. In certain embodiments, there is therefor provided herein a method for isolating GTA-446 from human serum, blood, or other suitable biological fluid comprising a step of solvent extraction/precipitation, followed by a step of column chromatographic separation.

In certain embodiments, there is provided herein a method for purifying GTA-446 from a biological fluid, said method comprising:
processing the biological fluid in a monophasic extraction/protein precipitation step, thereby producing a precipitated protein solid and a liquid phase;
separating the precipitated protein solid from the liquid phase;
processing the liquid phase in a phase separation step to obtain an organic serum extract;
processing the organic serum extract in a column separation step comprising:
   a normal phase separation step;
   a reverse phase separation step; and
   a two-stage HPLC separation step comprising a reverse phase stage followed by a normal phase stage;

wherein a purified GTA-446 fraction is eluted following the normal phase stage of the two-stage HPLC separation step, thereby providing purified GTA-446.

In certain embodiments of the above method, the monophasic extraction/protein precipitation step may comprise an extraction using ethyl acetate in water in the presence of methanol, where the methanol acts as a mediator for mixing the ethyl acetate in the water. By way of example, a monophasic extraction mixture comprising a solvent mixture having a ratio of about 1:2:4 of Serum:MeOH (with 1% Formic acid):EtOAc may be used for the monophasic extraction/protein precipitation step, which may involve allowing about 5 min wait time for precipitation to occur. In certain embodiments, the phase separation step may comprise the use of hexanes to phase separate the liquid phase, thereby obtaining the organic serum extract. In certain such embodiments, therefore, a solvent ratio at the phase separation step may be about 1:2:4:4 serum:methanol:ethyl acetate:hexane, for example.

Rationally Designed Labelled Derivatives

The discovery of the structure of formula I, and linking of this structure to the biomarker GTA-446, additionally allows for the rational design of isotopically labelled GTA-446 derivative compounds. Such compounds may, for example, be designed to facilitate mass spectrometry-based GTA-446 (or other GTA) analysis and/or quantification in biological samples. By way of example, isotopically labelled GTA-446 derivative compounds may be designed so as to provide internal standard signals corresponding to parent/daughter GTA-446 MS signals being used in the analysis and/or quantification method. Such isotopically labelled GTA-446 derivative compounds may, for example, be designed to provide internal standard signals corresponding to parent/daughter GTA-446 (or other GTA) MS signals being used in the analysis and/or quantification method, whereby the internal standard signals do not substantially overlap with, or receive interference from, other irrelevant serum signals detected during analysis.

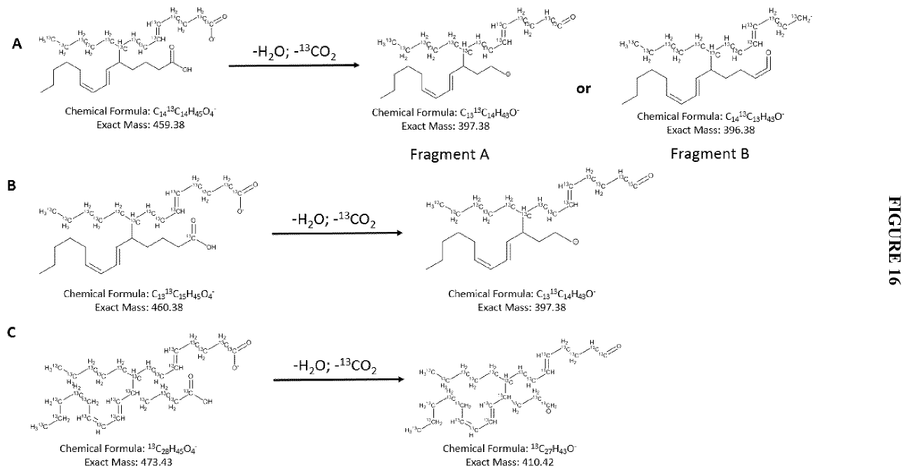

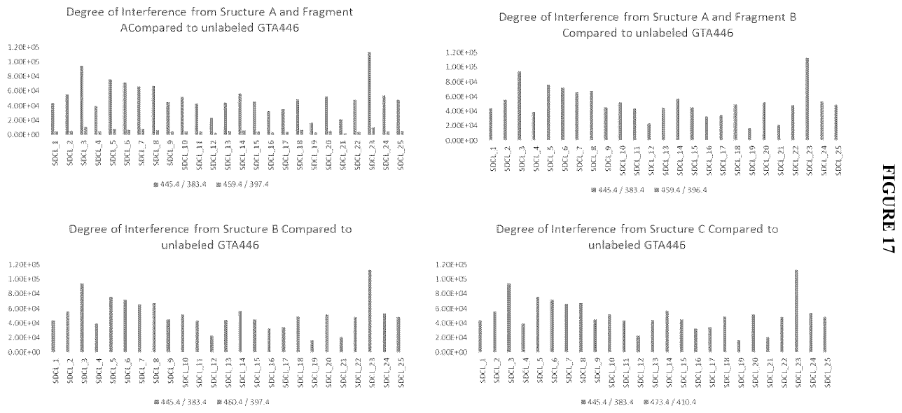

In designing examples of such isotopically labelled compounds, interference studies were performed using serum and tandem MS analysis to survey parent-daughter ion combinations of various theoretical ¹³C incorporated isoforms of GTA-446 to verify a lack of interfering signal. In certain examples, three candidate standards were analyzed which resulted in theoretical MS/MS fragments similar to corresponding unlabeled GTA-446 tandem MS pairs; and parent and daughter fragments after loss of water and carbon dioxide. See structures (A), (B), and (C) in FIG. 16, each of which represent rationally designed isotopically labelled GTA-446 derivatives. These were analyzed in 25 samples of individual serum extracts. Both molecules A and B, in particular, gave low interference in the un-spiked serum extracts, suggesting both as particularly useful potential internal standard candidates. Interference analysis of isotopically labelled compounds (A), (B), (C), and fragments A and B, as per FIG. 16 are shown in FIG. 17. Such isotopically labelled compounds may, in certain embodiments, be used for incorporation into stable isotope dilution methods for quantifying GTA-446 levels in serum.

In certain embodiments, there is provided herein an isotopically labelled compound, the isotopically labelled compound comprising one or more isotopic labels incorporated within the structure of formula I:

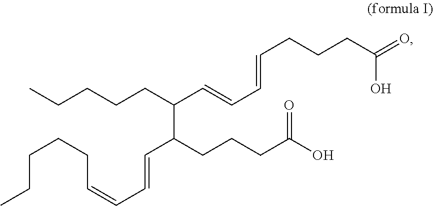 [SMILES: CCCCC/C=C\C=C\C(CCCC(=O)O)C(/C=C/C=C/CCCC(=O)O)CCCCC]

or a salt, ester, or prodrug thereof.

The isotopically labelled compound above may, in certain embodiments, comprise one or more isotopic labels which are stable isotope labels, radioisotope labels, or a combination thereof. The one or more isotopic labels may, for example, be selected from the group consisting of deuterium (²H) and ¹³C. The one or more isotopic labels may, in certain further embodiments, be selected from the group consisting of tritium (³H) and ¹⁴C.

In certain examples, the isotopically labelled compound may be or comprise a compound having the structure of formulas (A), (B), or (C):

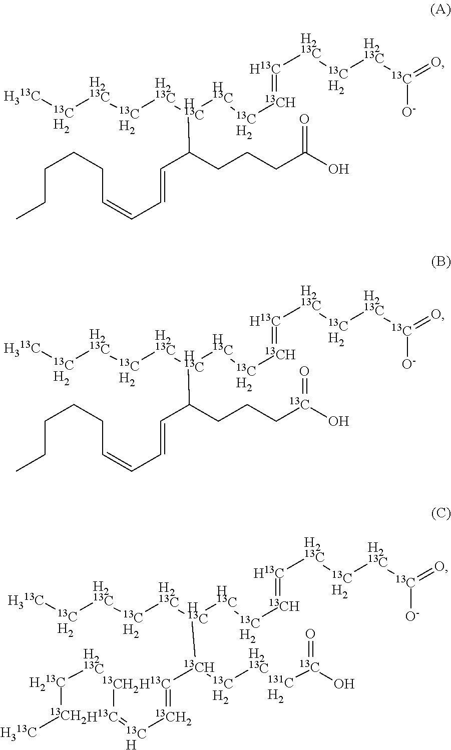

or a salt, ester, or prodrug thereof.

In certain embodiments, the isotopically labelled compound may apply the labelling strategies of any one of formulas (A), (B), or (C) to a compound of formula II, III, or IV.

In certain embodiments, the isotopically labelled compound may be for use as an analytical standard compound. In certain other embodiments, the isotopically labelled compound may be for use in a metabolic tracer composition, an in vitro or in vivo diagnostic agent, or in another composition.

Synthesis

In another embodiment, synthetically prepared GTA-446-related compounds (or salts, esters, or labelled derivatives thereof), or other compounds related thereto, as described herein are contemplated. In certain embodiments, such compounds, or derivatives thereof, are contemplated which have been prepared using a synthetic chemistry approach. Such synthetically prepared compounds may be used, for example, in an isotope-dilution mass spectrometry detection method.

Due to the difficulties in isolating GTA-446 from serum (yield of pure GTA446 is typically <1% from starting serum), and lack of a natural source of a stable isotopically labelled compound corresponding to GTA-446, synthetic production of unlabeled and/or labeled forms of GTA-446, or derivatives thereof, may be useful for facilitating GTA-446 assays such as stable isotopic assays.

Previously, to the best of our knowledge, there has been no known or reported isolation or synthetic scheme for obtaining GTA-446 or derivatives thereof. Given that the structure of GTA-446 was previously unknown, synthetic schemes for the preparation of compounds of formula I, and derivatives thereof, were simply not contemplated.

In the past, industrial-scale manufacturing of dicarboxylic fatty acid dimers utilizing monomeric, monounsaturated fatty acids has been reported for the production of lubricants and polymers. Such methods typically include harsh reaction conditions such as high temperature (200° C.-300° C.), high pressure (70-175 psi) and often use clay or clay mineral as a catalyst as well as prolonged mixing and agitation. The end products of these reactions typically comprise non-specific, polymeric mixtures. A typical schematic reaction pathway for a generalized fatty acid dimerization process based on such processes may comprise the following:

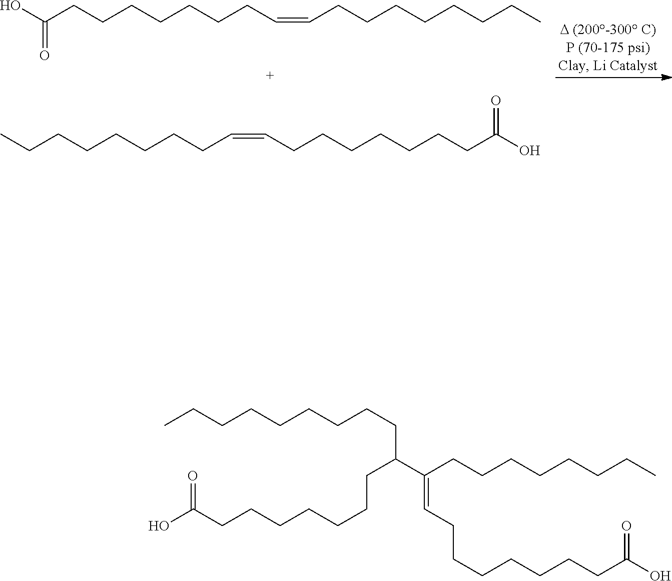

### 10-9′ Bridged Dimer of Stearic and Oleic Di-Carboxylic Acid

During the dimerization process, other side reactions such as cis/trans isomerization, double bond shift, branching, ring formation, and/or polymerization may also occur potentially leading to a variety of byproducts. Often a fraction distillation method is employed to separate desired species from the rest of the byproducts and unreacted starting material.

The abovementioned method may not be ideal for GTA-446 synthesis, as specificity for the location of the conjugating points and unsaturations may be difficult to achieve. Secondly, GTA-446 contains 4 double bonds, complicating the dimerization (monounsaturated fatty acids are shown in the example).

Thus, it is contemplated herein that a synthetic chemistry approach may be employed to produce compounds as described herein. Having regard to the teachings provided herein, including the elucidation of the GTA-446 structure, a variety of synthetic approaches may be contemplated by retrosynthetic analysis of the provided structures. Synthetic schemes may, in certain embodiments, involve the use of appropriate starting materials and/or the blocking/protection of reactive functional groups which substantially avoid rearrangement during synthesis to undesirable byproducts.

In certain embodiments, it is contemplated that synthetic approaches may involve, for example, carbon-carbon (C—C) dimerization between two C₁₄ carboxylic acid chains, retaining the specific alkene stereochemistry by using appropriate catalysts and/or by blocking of reactive sites to achieve the desired end-product. Suitable asymmetric Heck coupling, or clay mineral catalyzed high temperature/high pressure dimerization, for example, may also be employed to synthesize GTA-446.

Isotopically labelled derivatives of GTA-446 may be synthesized either as deuterium (²H) or ¹³C forms, for example. Deuterium may undergo hydrogen exchange with the solvent under certain conditions, giving an equilibrium between the deuterated and the non-deuterated form. ¹³C is a particularly stable isotope form with low natural abundance (˜1%), which would not be expected to undergo substantial rearrangements with its ¹²C versions since it is covalently incorporated by C—C bonds. Isotopically labelled compounds comprising at least one deuterium, at least one ¹³C, or both, are all contemplated herein, and may be selected to suit a particular application. For example, where high stability is desired, ¹³C may be used, however deuterium-labelled derivatives are also contemplated for certain applications as well. Radiolabelled derivatives of compounds described herein are also contemplated, depending on the particular application. In certain embodiments, labelled derivatives may be generated by using one or more labelled reagents/reactants during synthesis, as described in further detail hereinbelow.

Synthetic strategies for preparing GTA compounds, and derivatives thereof, are set out below, and more detailed proposed synthetic routes for preparing compounds of formulas I and III are further described in Examples 4 and 5 below.

The following synthetic routes may generate GTA compounds, derivatives thereof, and/or compounds related thereto from a precursor compound of formula D, or a labelled derivative thereof:

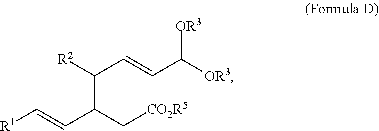

wherein:
R¹ is —Sn(R¹⁰)₃, —OTf, —Cl, —Br, —I, —B(OH)₂ or

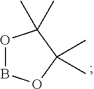

R² is optionally substituted saturated or unsaturated C₁-C₂₀ alkyl, saturated or unsaturated C₂-C₂₀ alkenyl, or saturated or unsaturated C₂-C₂₀ alkynyl;
each R³ is, independently, optionally substituted C₁-C₆ alkyl, or the R³ groups together form an optionally substituted ethylene or propylene group bridging the attached oxygen atoms to form a five- or six-membered ring;
R⁵ is optionally substituted C₁-C₆ alkyl; and
R¹⁰ is optionally substituted C₁-C₆ alkyl.

In certain embodiments, it is contemplated that compounds of formula D may be used to generate compounds of formula F, G, or H by reaction with a compound of formula E as follows:

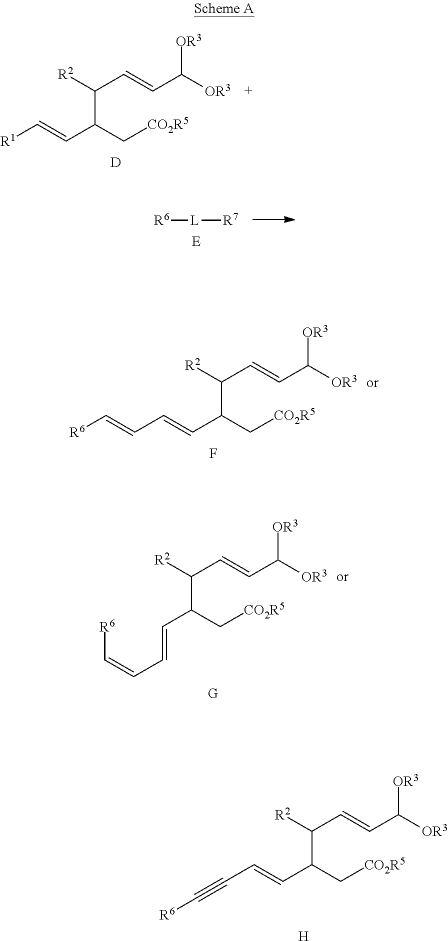

As will be understood, compounds D and E may be coupled using Stille, Sonagashira, Suzuke or any other suitable metal-mediated cross-coupling reaction, so as to provide at least one of compound F, compound G or compound H. The skilled person having regard to the teachings herein will recognize that alternate cross-coupling reactions may be useful in coupling compound A with compound B. The compound of formula E (and, likewise, the R¹ moiety of formula D) may be selected so as to be compatible for such coupling reaction.

In certain embodiments, the compound of formula E may include those in which:
R⁶ is optionally substituted saturated or unsaturated C₁-C₂₀ alkyl, saturated or unsaturated C₂-C₂₀ alkenyl, or saturated or unsaturated C₂-C₂₀ alkynyl;
R⁷ is —H, —Sn(R¹⁰)³, —OTf, —Cl, —Br, —I, —B(OH)₂ or

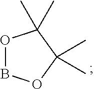

L is

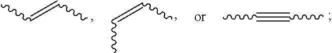

and
R¹⁰ is optionally substituted C₁-C₆ alkyl;
wherein R⁷ and L are selected for reaction with the vinyl R1 group of formula D to yield any one of compounds of formula F, G, or H.

It is contemplated that a compound of formula F, G, or H may then be used to generate GTA compounds, derivatives thereof, and/or compounds related thereto.

Where a compound of formula F is used, it is contemplated that a GTA compound, derivative thereof, and/or compound related thereto may be prepared as follows:

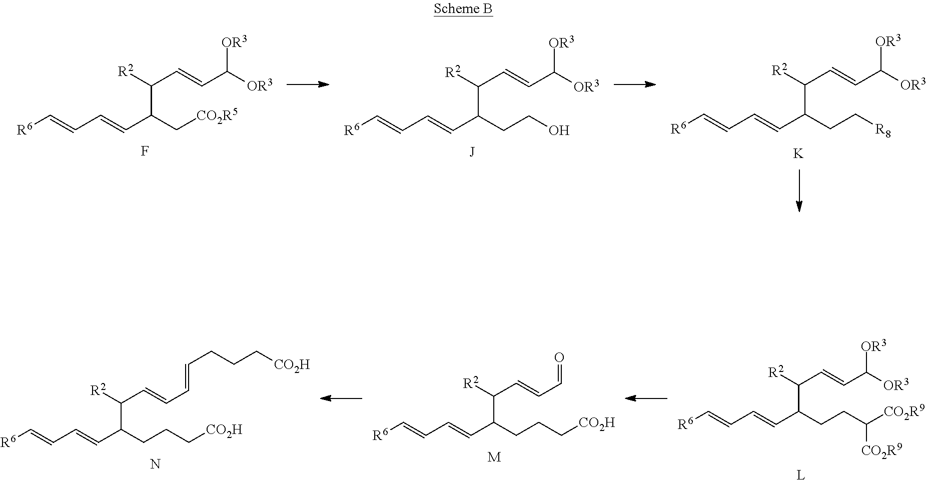

It is contemplated that Compound J may be prepared by reduction of the ester of compound F, for example using lithium aluminum hydride, lithium borohydride or DIBAL. The person of skill in the art having regard to the teachings herein will recognize that there are alternate reagents that may be useful for the reduction of an ester to an alcohol. Compound K may be prepared by reacting compound J with MsCl, Ts-Cl, Bs-Cl, triflic anhydride, CBr₄/PPh₃, N-iodosuccinimide/PPh₃, or CCl₄/PPh₃, for example. The person of skill in the art having regard to the teachings herein will recognize that alternate conditions may also be suitable for converting an alcohol to a leaving group. In the compound of formula K, R⁸ may be —OMs, —OTf, —OTs, —OBs, —Cl, —Br, —I or any other suitable leaving group, depending on the particular application. Compound L may be prepared by reacting compound K with a dialkyl malonate under basic conditions, for example. The person of skill in the art having regard to the teachings herein will recognize that there may be many suitable conditions for displacing a leaving group with a dialkyl malonate. In the compound of formula L, each R⁹ may be, independently, an optionally substituted C₁-C₆ alkyl, for example. It is contemplated that compound M may be prepared by simultaneous acid hydrolysis of an acetal and an ester with tandem decarboxylation of compound L. The person of skill in the art having regard to the teachings herein will recognize that there are various reagents and catalysts that may be useful for this transformation. Compound N may be prepared by Wittig reaction with compound M. The person of skill in the art having regard to the teachings herein will recognize that Wittig reaction alternatives may be available and may include, but are not limited to, Julia coupling and Horner-Emmons coupling reactions.

Where a compound of formula G or H is used, it is contemplated that a GTA compound, derivative thereof, and/or compound related thereto may be prepared as follows:

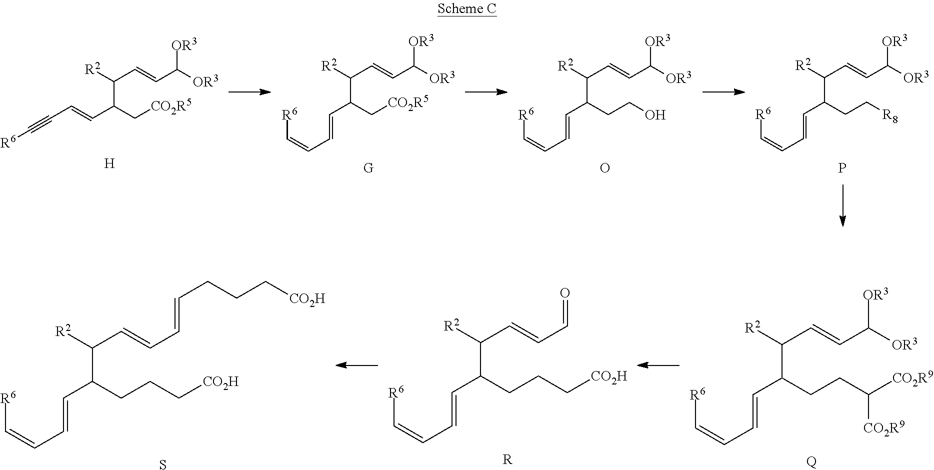

It is contemplated that compound H may be converted to compound G by reduction using reagents such as Lindlar's catalyst or borane (hydroboration). The person of skill in the art having regard to the teachings herein will recognize that suitable alternate reagents and methods for affecting reduction of a triple bond to a cis-olefin may also be used. Compound O may be prepared by reduction of the ester of compound G using lithium aluminum hydride, lithium borohydride or DIBAL. The person of skill in the art having regard to the teachings herein will recognize that alternate reagents may be useful for the reduction of an ester to an alcohol. Compound P may be prepared by reacting compound O with MsCl, Ts-Cl, Bs-Cl, triflic anhydride, CBr₄/PPh₃, N-iodosuccinimide/PPh₃, or CCl₄/PPh₃, for example. The person of skill in the art having regard to the teachings herein will recognize that alternate conditions useful for converting an alcohol to a leaving group may be used. In the compound of formula P, R⁸ may be —OMs, —OTf, —OTs, —OBs, —Cl, —Br, —I or any other suitable leaving group, depending on the particular application. Compound Q may be prepared by reacting compound P with a dialkyl malonate under basic conditions. The person of skill in the art having regard to the teachings herein will recognize that there may be many available conditions for displacing a leaving group with a dialkyl malonate. In the compound of formula Q, each R⁹ may be, independently, an optionally substituted C₁-C₆ alkyl, for example. Compound R may be prepared by simultaneous acid hydrolysis of an acetal and an ester with tandem decarboxylation of compound Q. The person of skill in the art having regard to the teachings herein will recognize that there may be various reagents and catalysts that may be useful for this transformation. It is contemplated that compound S may be prepared by Wittig reaction with compound R. The person of skill in the art having regard to the teachings herein will recognize that Wittig reaction alternatives may be available and may include, but are not limited to, Julia coupling and Horner-Emmons coupling reactions.

As will be understood, synthetic routes such as those set out above may be used to generate GTA compounds, derivatives thereof, labelled versions thereof, and/or compounds related thereto. Through selection of reagents, reactants, and R groups, it is contemplated that a variety of different GTA or GTA-like compounds may be prepared using such synthetic routes. In certain embodiments, labels, such as isotopic label(s) and/or radiolabel(s), may be incorporated into such GTA compounds and derivatives by using reagents/reactants carrying one or more label(s) such that the label(s) become incorporated into the compounds during synthesis.

More detailed proposed synthetic routes for preparing compounds of formulas I and III are further described in Examples 4 and 5, provided below.

Methods for Determining a Level of a GTA in a Sample

GTA-446 compounds, and derivatives thereof including isotopically labelled derivatives, may be particularly useful in GTA quantitation assays. In certain embodiments, such compounds may be used for enhancing the commercially available Cologic® colon cancer screening test by allowing for GTA-446 quantitation including the use of isotope-dilution mass spectrometry methods, for example.

Examples of GTA-446 quantification methods and assays, and diagnostic methods and assays for identifying a subject as having, or being at risk of developing, colorectal cancer, may include those which are described in detail in PCT application publication no. WO 2007/030928, which is herein incorporated by reference in its entirety. The reference describes, among other things, a metabolic marker of 446.34 Daltons, corresponding to GTA-446 and the newly elucidated structures described herein.

As will be understood, GTA-446 compounds, and derivatives thereof including isotopically labelled derivatives as described herein, may be useful in GTA quantitation assays investigating levels of a gastric tract acid (GTA) of interest. It will be recognized that gastric tract fatty acids (GTAs) comprise a family of homo- and hetero-14 to 22 carbon dimeric fatty acids, and it is contemplated herein that the presently described GTA-446 compounds, and derivatives thereof, may be useful in the quantitation of these GTAs.

A prototypical GTA family member, GTA-446, consists of two 14-carbon chains as depicted by formula I. GTA-446 is the analyte measured in the commercial Cologic™ blood test for colorectal cancer screening, while the GTA PC-594, a 36 carbon dicarboxylic fatty acid comprised of two dimerized 18-carbon fatty acids, is measured in the PanaSee™ blood test for pancreatic cancer risk. Currently, neither method incorporates internal standards, due to the lack of such standard. The lack of such standards makes it difficult to run the assay on multiple platforms, and somewhat limits the assay results. The presently described subject-matter may be used to address the lack of an internal standard, by providing ¹²C and ¹³C structures which may be suitable for combination with assays such as Cologic™ and PanaSee™

Although GTA-446 specific standards may be particularly well-suited for the Cologic™ assay which measures GTA-446 levels, it is contemplated that such standards may also be useful for the quantification of any other suitable GTA. All GTAs measured to date show a unique fragmentation pattern under collision-induced dissociation (CID) tandem mass spectrometry, particularly in the relative pattern of water and carbon dioxide losses. Without wishing to be bound by theory, one skilled in the art will appreciate that an alternate reference standard may be used to quantify a variety of molecules, so long as the standard exhibits similar properties as the target analyte. In the case of the GTA metabolic system, the behavior under CID of GTA-446 is similar to other GTAs, and thus a ¹³C internal standard of GTA-446 and an isotope dilution curve of ¹²C/¹³C GTA446 may be of use to provide relative quantification across multiple GTA species with a suitable degree of analytical confidence.

In certain embodiments, GTA-446-based compounds as described herein may be used in combination with, for example, the PanaSee™ assay to investigate GTA PC-594. By way of example, GTA-446-based compounds as described herein may be used to investigate levels of a GTA as described in WO 2011/038509. Thus, in certain embodiments, there is provided herein a method for determining a level of a gastric tract acid (GTA) in a sample, said method comprising:
measuring a GTA detection signal from the sample, the GTA detection signal representative of the GTA level in the sample; and
quantifying the level of the GTA in the sample by comparing the measured GTA detection signal with a calibration reference.

In certain embodiments, the GTA may be:

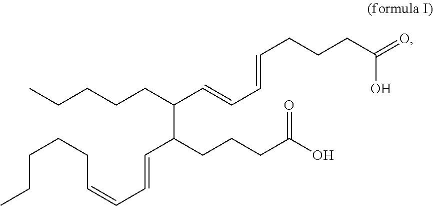 [SMILES: CCCCC/C=C\C=C\C(CCCC(=O)O)C(/C=C/C=C/CCCC(=O)O)CCCCC]

or another suitable GTA family member.

In certain embodiments, the GTA detection signal may be measured by mass spectrometry. In certain further embodiments, the GTA detection signal may be measured by mass spectrometric techniques as per the commercially available Cologic® assay and/or as per techniques as described in PCT application publication no. WO 2007/030928, which is herein incorporated by reference in its entirety.

In certain embodiments of the above-described methods, the calibration reference may comprise a standard curve prepared using known quantities of a compound as defined herein.

In certain embodiments, the method may further comprise a step of:
spiking the sample with a known quantity of an isotopically labelled compound as
defined herein; and
measuring an internal standard signal from the sample, the internal standard signal being representative of the known quantity of the isotopically labelled compound spiked into the sample.

The internal standard signal may also, in certain embodiments, be measured by mass spectrometry.

In certain embodiments, the methods described above may further comprise a step of determining a ratio of the GTA level in the sample, as represented by the measured GTA detection signal, to the known quantity of isotopically labelled compound spiked into the sample, as represented by the internal standard signal. The calibration reference may, in certain embodiments, comprise an isotope dilution curve (IDC) generated from a series of mixtures of varying GTA/isotopically labelled compound ratios and concentrations, to which said ratio is compared. In certain embodiments, the IDC may be generated from a series of mixtures in which GTA content is varied over a fixed amount of isotopically labelled compound. In certain embodiments, the fixed amount of the isotopically labelled compound may be substantially the same as the known quantity of the isotopically labelled compound which is spiked into the sample.

The skilled person having regard to the teachings herein will be aware of IDC mass spectrometric techniques, isotope-dilution mass spectrometry techniques, and methods for generating IDC curves, and will be able to select and/or modify such techniques as desired to suit a particular application.

There is also provided herein a use of a compound as described herein for generating a calibration reference for use in determining a level of a gastric tract acid (GTA) in a sample. In certain embodiments, the GTA may be:

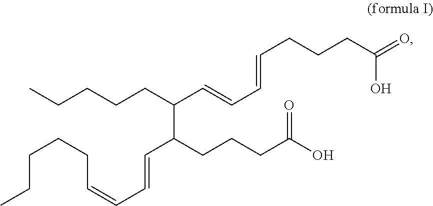 [SMILES: CCCCC/C=C\C=C\C(CCCC(=O)O)C(/C=C/C=C/CCCC(=O)O)CCCCC]

or another suitable GTA family member.

As well, there is provided herein a use of an isotopically labelled compound as defined herein as an internal standard for use in determining a level of a gastric tract acid (GTA) in a sample. In certain embodiments, the GTA may be:

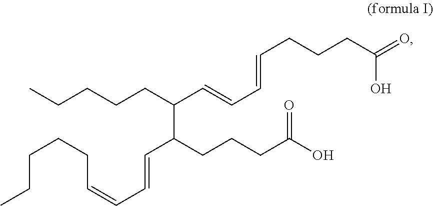 [SMILES: CCCCC/C=C\C=C\C(CCCC(=O)O)C(/C=C/C=C/CCCC(=O)O)CCCCC]

or another suitable GTA family member.

As described herein, quantifying or quantification is intended to relate to a determination of the amount of a particular molecule, e.g. a GTA, in a sample or body fluid in either relative or quantitative terms. For example, this may mean the determination of the concentration of the molecule in moles/L, percent by weight, or other standard unit of measurement. Alternatively, the terms may relate to a relative determination of the level of the molecule with respect to an internal standard or a control (e.g. without calculating the concentration). For instance, a relative quantification of the molecule may involve the determination of an increase or decrease in a subject as compared to the internal standard or control, e.g over time as compared to previous timepoints to measure progression of disease or treatment.

Diagnostic Methods for Identifying a Subject as Having, or being at Risk of Developing, Colorectal Cancer

GTA-446 compounds, and derivatives thereof including isotopically labelled derivatives, may be particularly useful in diagnostic methods for identifying a subject as having, or being at risk of developing, a colorectal cancer which is linked to GTA-446 levels. In certain embodiments, such compounds may be used for enhancing the commercially available Cologic® colon cancer screening test by allowing for GTA-446 quantitation including the use of isotope-dilution mass spectrometry methods, for example.

Examples of GTA-446 quantification methods and assays, and diagnostic methods and assays for identifying a subject as having, or being at risk of developing, colorectal cancer, may include those which are described in detail in PCT application publication no. WO 2007/030928, which is herein incorporated by reference in its entirety. The reference describes, among other things, a metabolic marker of 446.34 Daltons, corresponding to GTA-446 and the newly elucidated structures described herein.

In certain embodiments, there is provided herein a diagnostic method for identifying a subject as having, or being at risk of developing, colorectal cancer, said method comprising:
determining a level of a gastric tract acid (GTA) in a sample obtained from the subject by
   measuring a GTA detection signal from the sample, the GTA detection signal representative of the GTA level in the sample; and
   quantifying the level of the GTA in the sample by comparing the measured GTA detection signal with a calibration reference, and
identifying the subject as having, or being at risk of developing, colorectal cancer when the determined level of the GTA in the sample is reduced in comparison to a healthy control group,

wherein the GTA is:

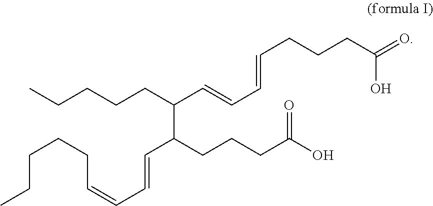 [SMILES: CCCCC/C=C\C=C\C(CCCC(=O)O)C(/C=C/C=C/CCCC(=O)O)CCCCC]

In certain embodiments, the GTA detection signal may be measured by mass spectrometry. In certain further embodiments, the GTA detection signal may be measured by mass spectrometric techniques as per the commercially available Cologic® assay and/or as per techniques as described in PCT application publication no. WO 2007/030928, which is herein incorporated by reference in its entirety.

In certain embodiments of the above-described methods, the calibration reference may comprise a standard curve prepared using known quantities of a compound as defined herein.

In certain embodiments, the step of determining the level of the GTA in the sample obtained from the subject may further comprise:
spiking the sample with a known quantity of an isotopically labelled compound as described herein; and
measuring an internal standard signal from the sample, the internal standard signal being representative of the known quantity of the isotopically labelled compound spiked into the sample.

In certain embodiments, the internal standard signal may also be measured by mass spectrometry.

In certain embodiments, such methods may further comprise a step of determining a ratio of the GTA level in the sample, as represented by the measured GTA detection signal, to the known quantity of isotopically labelled compound spiked into the sample, as represented by the internal standard signal. The calibration reference may, in certain embodiments, comprise an isotope dilution curve (IDC) generated from a series of mixtures of varying GTA/isotopically labelled compound ratios and concentrations, to which said ratio is compared. In certain embodiments, the IDC may be generated from a series of mixtures in which GTA content is varied over a fixed amount of isotopically labelled compound. The fixed amount of the isotopically labelled compound may, in certain embodiments, be substantially the same as the known quantity of the isotopically labelled compound which is spiked into the sample.

The skilled person having regard to the teachings herein will be aware of IDC mass spectrometric techniques, isotope-dilution mass spectrometry techniques, and methods for generating IDC curves, and will be able to select and/or modify such techniques as desired to suit a particular application.

In certain embodiments, there is provided herein a use of a compound as described herein in a diagnostic method for identifying a subject as having, or being at risk of developing, a colorectal cancer associated with altered levels of a gastric tract acid (GTA) which is:

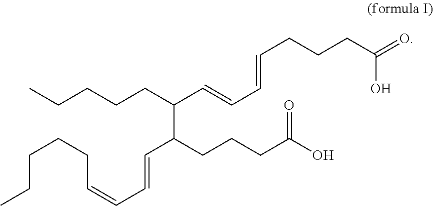 [SMILES: CCCCC/C=C\C=C\C(CCCC(=O)O)C(/C=C/C=C/CCCC(=O)O)CCCCC]

In another embodiment, there is provided herein a use of a compound as described herein for generating a calibration reference for use in a diagnostic method for identifying a subject as having, or being at risk of developing, a colorectal cancer associated with altered levels of a gastric tract acid (GTA) which is:

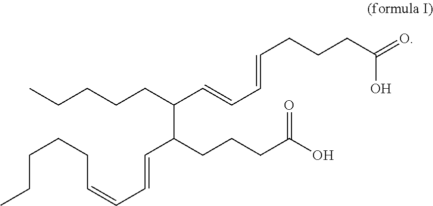 [SMILES: CCCCC/C=C\C=C\C(CCCC(=O)O)C(/C=C/C=C/CCCC(=O)O)CCCCC]

In yet another embodiment, there is provided herein a use of an isotopically labelled compound as described herein as an internal standard for use in a diagnostic method for identifying a subject as having, or being at risk of developing, a colorectal cancer associated with altered levels of a gastric tract acid (GTA) which is:

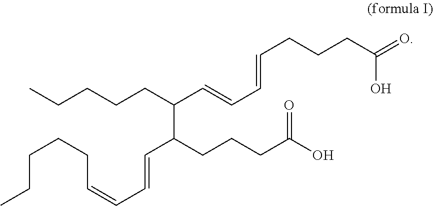 [SMILES: CCCCC/C=C\C=C\C(CCCC(=O)O)C(/C=C/C=C/CCCC(=O)O)CCCCC]

Immunoassays—Antibody Generation

In another embodiment, there is provided herein a use of GTA-446 compounds as described herein for generating antibodies, or fragments thereof, which specifically bind GTA-446. Such antibodies may have a variety of uses in the detection and/or quantification of GTA-446. Such antibodies may, in certain embodiments, be for use in immunoassays, such as enzyme-linked immunosorbent assays (ELISAs), for detecting and/or quantitating GTA-446 levels in a sample.

Previously, quantification of GTA-446 has been limited to tandem mass spectrometry, and has not been performed by enzyme-linked immunosorbent assay (ELISA), due to a lack of a suitable antibody. The production of a specific antibody generally requires sufficient quantities of pure compound, which has previously been limited by the unavailability of synthetic GTA-446.

In certain embodiments, there is provided herein a use of an isolated, purified, or synthetic GTA-446 in the generation of an anti-GTA-446 antibody. In certain further embodiments, there is provided herein a GTA-446 detection and/or quantification immunoassay which employs such an anti-GTA-446 antibody.

In an embodiment, there is provided herein an antibody, or antigen-binding fragment thereof, which specifically binds to a compound of formula I:

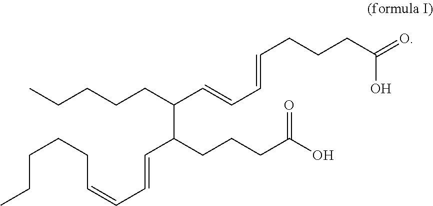 [SMILES: CCCCC/C=C\C=C\C(CCCC(=O)O)C(/C=C/C=C/CCCC(=O)O)CCCCC]

In certain embodiments, the antibody may be or comprise a monoclonal or polyclonal antibody.

In another embodiment, there is provided herein a use of a compound as described herein as an antigen for preparing an antibody which specifically binds to an antigenic epitope of said compound.

The person of skill in the art having regard to the teachings herein will be aware of a variety of suitable techniques for generating anti-GTA-446 antibodies, or fragments thereof, as described herein. Examples of such techniques may include those described in Antibodies: A Laboratoy Manual, 2^{nd} Ed., Cold Spring Harbor Laboratory Press, 2014, ISBN: 978-1-936113-81-1; which is herein incorporated by reference in its entirety.

Immunoassays for Determining a Level of a GTA in a Sample

Anti-GTA-446 antibodies as described herein may be used in immunoassays, such as but not limited to ELISA-based assays, for the detection and/or quantification of GTA levels in a sample.

In certain embodiments, there is provided herein a method for determining a level of a gastric tract acid (GTA) in a sample, said method comprising:
measuring a level of the GTA in the sample using an immunoassay employing an antibody, or antigen-binding fragment thereof, which specifically binds to the GTA;

wherein the GTA is:

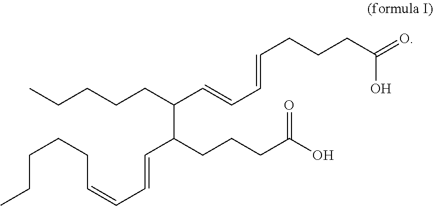 [SMILES: CCCCC/C=C\C=C\C(CCCC(=O)O)C(/C=C/C=C/CCCC(=O)O)CCCCC]

In certain embodiments, the immunoassay may comprise an enzyme-linked immunosorbent assay (ELISA).

In certain embodiments, the method may further comprise a step of using a control sample comprising a compound as defined herein as a positive control in the immunoassay.

In a further embodiment, the method may further comprise a step of using a standard curve to extrapolate the level of the GTA in the sample, the standard curve having been generated using a plurality of known quantities of a compound as described herein.

In another embodiment, there is provided herein a use of an anti-GTA-446 antibody, or fragment thereof, for detecting or quantifying a level of a gastric tract acid (GTA) in a sample by immunoassay, wherein the GTA is:

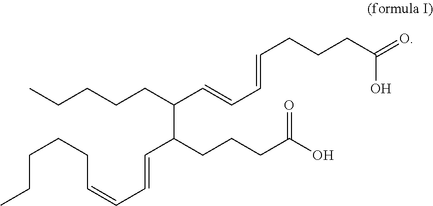 [SMILES: CCCCC/C=C\C=C\C(CCCC(=O)O)C(/C=C/C=C/CCCC(=O)O)CCCCC]

Immunoassays for Identifying a Subject as Having, or Being at Risk of Developing, Colorectal Cancer

Anti-GTA-446 antibodies as described herein may be used in immunoassays, such as but not limited to ELISA-based assays, for the detection and/or quantification of GTA levels in a sample as part of a diagnostic method identifying a subject as having, or being at risk of developing, colorectal cancer (CRC).

In certain embodiments, there is provided herein a diagnostic method for identifying a subject as having, or being at risk of developing, colorectal cancer, said method comprising:
determining a level of a gastric tract acid (GTA) in a sample obtained from the subject by
   measuring a level of the GTA in the sample using an immunoassay employing an antibody, or antigen-binding fragment thereof, which specifically binds to the GTA; and
identifying the subject as having, or being at risk of developing, colorectal cancer when the determined level of the GTA in the sample is reduced in comparison to a healthy control group,

wherein the GTA is:

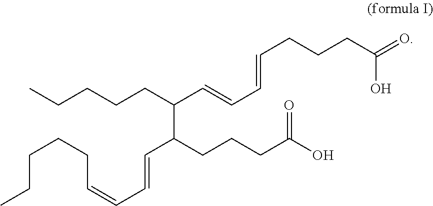 [SMILES: CCCCC/C=C\C=C\C(CCCC(=O)O)C(/C=C/C=C/CCCC(=O)O)CCCCC]

In certain embodiments, the immunoassay may comprise an enzyme-linked immunosorbent assay (ELISA).

In a further embodiment, the method may further comprise a step of using a control sample comprising a compound as described herein as a positive control in the immunoassay.

In a further embodiment, the step of determining the level of the GTA in the sample may further comprise using a standard curve to extrapolate the level of the GTA in the sample, the standard curve having been generated using a plurality of known quantities of a compound as described herein.

In another embodiment, there is provided herein a use of an anti-GTA-446 antibody, or an antigen-binding fragment thereof, as described herein in a diagnostic method for identifying a subject as having, or being at risk of developing, a colorectal cancer associated with altered levels of a gastric tract acid (GTA) which is:

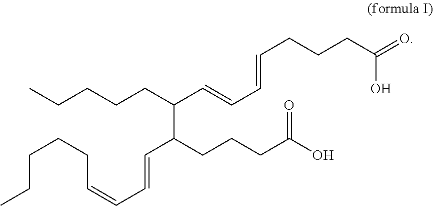 [SMILES: CCCCC/C=C\C=C\C(CCCC(=O)O)C(/C=C/C=C/CCCC(=O)O)CCCCC]

Quantification and/or Diagnostic Kits

In certain embodiments, there are provided herein kits relating to the detection and/or quantification of GTA levels in a sample.

In an embodiment, there is provided herein a kit for quantifying a level of a gastric tract acid (GTA) in a sample, said kit comprising at least one of:
a compound as described herein;
a metabolic tracer as described herein;
a composition as described herein;
a diagnostic agent as described herein; and
an antibody, or antigen-binding fragment thereof, as described herein;
and, optionally, further comprising a set of instructions for performing a method as described herein.

In another embodiment, there is provided herein a diagnostic kit for identifying a subject as having, or being at risk of developing, colorectal cancer, the kit comprising at least one of: a compound as described herein;
a compound as described herein;
a metabolic tracer as described herein;
a composition as described herein;
a diagnostic agent as described herein; and
an antibody, or antigen-binding fragment thereof, as described herein;
and, optionally, further comprising a set of instructions for performing a method as described herein.

### Example 1—Methods for GTA-446 Isolation from Human Serum

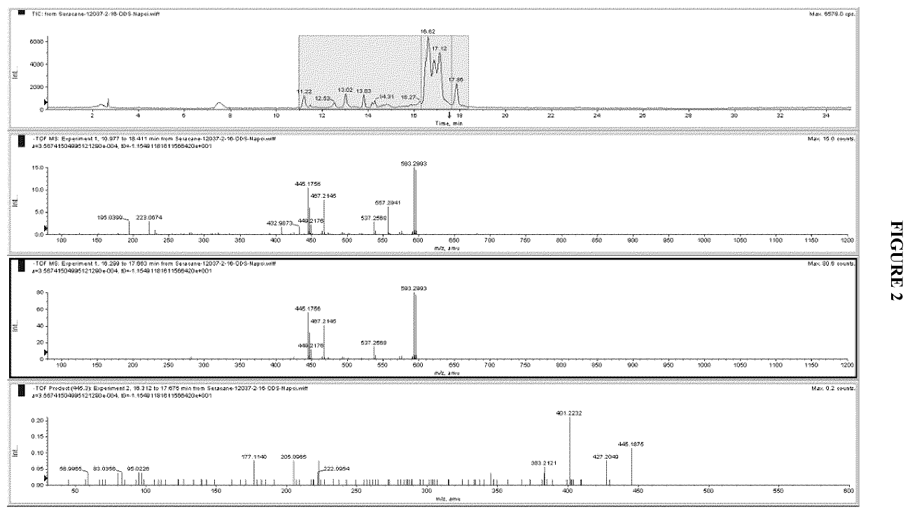

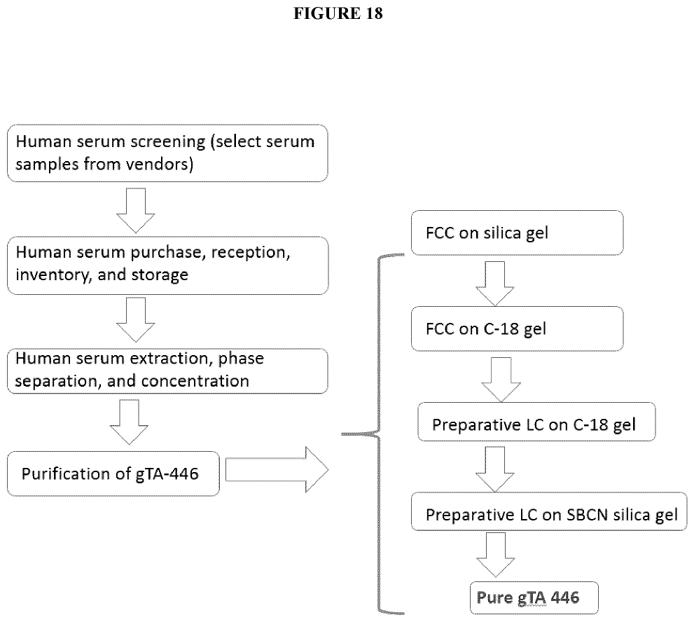

A general process for isolating GTA-446 (see, for example, FIG. 18) from human serum may involve the evaluation of multiple lots of commercially available serum for a lot with high GTA-446 concentration, followed by the procurement of large (50 L) quantities of the chosen lot. The test lots of serum in this example were extracted between water and ethyl acetate buffered with formic acid. GTA-446 eluted with a pool of nine other similar fatty acids between 16.3-17.6 minutes under the selected method (see FIG. 2). Selection of the serum source was decided based on how clean and enriched the staring extraction with GTA-446 was for a scale up extraction since other contaminants may reduce the efficiency of purification in the process.

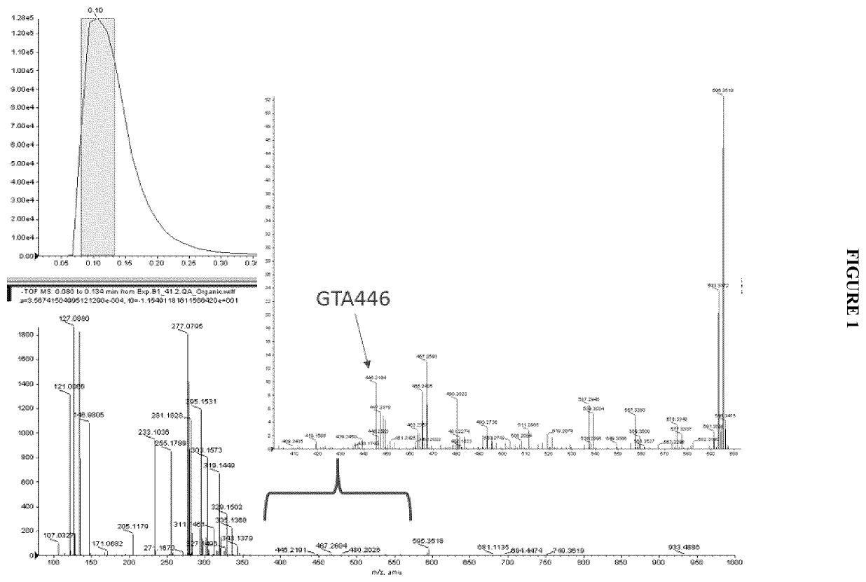

Due to the low abundance of endogenous GTA-446 in serum (FIG. 1), a serum extraction protocol was designed herein. Human serum matrix is composed of a variety of components that have a range of degrees of solubility. In addition to complex molecules such as proteins, lipids and carbohydrates, serum also contains very small molecules such as amino acids, vitamins and other small metabolites. Their structural functionalities result in varying degrees of solubility, ranging from very polar (carbohydrates and proteins and other hydrophilic components in water/methanol media) to medium polar (phospholipids and fatty acids and other heteroatomic small metabolites in ethyl acetate, chloroform and dichloromethane) to non-polar (tri and di glycerides and larger glycolipids in hexane) solvent combinations. Therefore, selection of an appropriate solvent scheme for the initial separation step to obtain a GTA-446 enriched fraction that was safe and convenient to handle and evaporate was performed. The tandem MS/MS fragmentation, together with the low molecular weight (<500 amu), suggested that the target molecule was a small metabolite with carboxylic and hydroxyl functionalities (as described above), and would be readily solubilize in an ethyl acetate/methanol solvent combination. This solvent combination is convenient for the extraction, since methanol results in the precipitation of the proteins in the matrix.

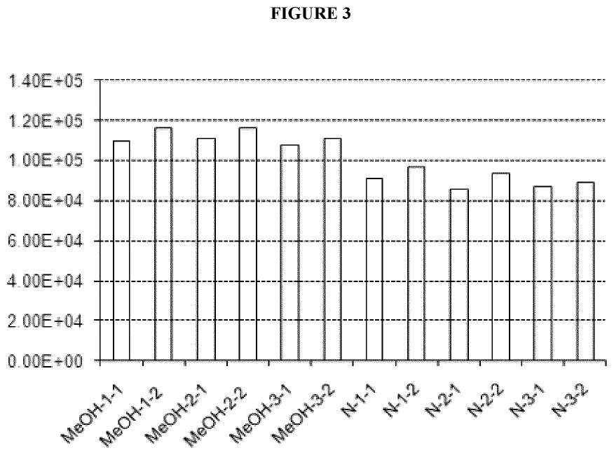

Due to miscibility of ethyl acetate in water in the presence of methanol (the latter acting as a mediator for mixing ethyl acetate in water), a monophasic precipitation step was designed to achieve an good concentration of GTA-446 in the preliminary GTA-446 enrichment step. Unlike a biphasic precipitation, where complete migration of the CRC markers into the organic phase might be inhibited due to weak electrostatic attractions and intermolecular hydrogen bonding with the aqueous phase, the monophasic precipitation step reduced such risk since there is not any initial phase separation. To verify this hypothesis, extracts between monophasic (MeOH-1 to MeOH-2) and biphasic (N1-N3) separation were compared using MS analysis, which detected that the former shows a 20% extraction efficiency over the latter (FIG. 3).

Selection of a methanol/ethyl acetate ratio with proper acid strength, and a wait time for the precipitation, were studied in an effort to improve GTA-446 extraction. The key focuses during method development in this step were to obtain good extraction efficiency using low solvent volumes, which would also allow reduction of the evaporation time as well as solvent waste, and to use a low acid strength. A solvent mixture having the ratio of 1:2:4 of Serum:MeOH (with 1% Formic acid):EtOAc was selected for the precipitation step, allowing 5 min wait time for the precipitation.

The precipitated protein solids were separated from the liquids by centrifuging and decanting the homogenized liquids, now having all the polar and nonpolar solubles in serum. The next stage in the isolation process was the phase separation of GTA-446 into a volatile solvent system to facilitate low temperature evaporation under reduced pressure and to obtain a crude organic extract of serum containing GTA-446. In the monophasic solution, both organic and the aqueous phases are inseparable since methanol acts as an intermediate to mix water and ethyl acetate.

Since the presence of water in the extraction media produced longer times and higher temperatures during the evaporation step, large volumes of solvent mixture (H₂O:MeOH:EtOAc , 1:2:4) were inconvenient to evaporate using small scale rota-vapors efficiently. Also, the stability of GTA-446 in acidic aqueous phase was a concern due to possible formation of lactones. Therefore, a quick and efficient separation of GTA-446 from aqueous phase into a neutral, less polar, organic phase was designed by introducing a phase separation step into the extraction sequence subsequent to the monophasic precipitation step. This also separated most of the unwanted polar substances extracted into the aqueous phase from the analyte of interest. The phase separation was achieved either by adjusting the solvent ratios between water and ethyl acetate; a higher volume of ethyl acetate over water easily separates the solvents into two phases; or by adding a non-miscible non polar solvent, such as hexane, which disperses the organic solubles in the upper organic phase. A series of experiments were designed to have additions of different volumes of water, ethyl acetate and hexane in different combinations into the solvent mixture obtained from the precipitation step (Table 1).

TABLE 1
Different test batches used to investigate the
solvent ratios in the phase separation step

| | Solvents used in | | | | | |
|---|---|---|---|---|---|---|
| | precipitation step/ml | | | Solvents used in | | |
| | | 0.5% FA | | phase separation/ml | | |
| Expt # | Serum | in MeOH | EtOAc | Water | EtOAc | hexane |
| 1 | 5 | 10 | 20 | 40 | 20 | — |
| 2 | 5 | 10 | 20 | 40 | — | 20 |
| 3 | 5 | 10 | 20 | — | — | 20 |

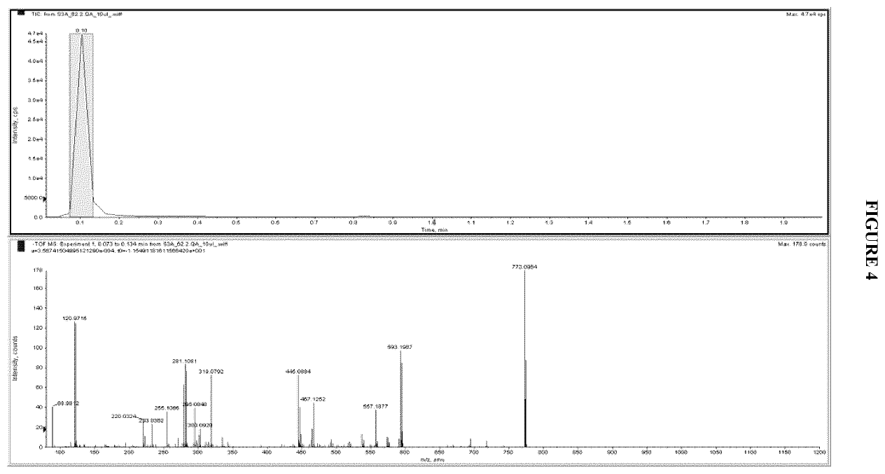

Both organic and aqueous phases were collected and analyzed by time of flight mass spectrometry to determine the GTA-446 extraction efficiency in each experiment. The GTA marker panel was detected in the organic phases of all the types of test extractions showing no noticeable difference in the extraction efficiency. Since there was no discrimination on GTA-446 extraction among the three solvent combinations, hexane was selected due to superior clarity of the phase separation. In the two experiments where water was added to obtain the phase separation, an emulsion was formed which took a longer time to give a clear separation. Also, the total volume of the aqueous phase was much less compared to the other two in the bulk extraction, and was easy to handle. Therefore the final solvent ratio identified was 1:2:4:4 serum:methanol:ethyl acetate:hexane. The organic upper layer was then evaporated to dryness to obtain the crude extracts enriched for GTA-446, devoid of most of the polar impurities in its original matrix (see FIG. 4).

The next step of the isolation process was the separation and purification of GTA-446 from other impurities in the crude matrix, and relied on a column separation sequence using normal and reverse phase high performance liquid chromatography (HPLC), as well as flash column chromatography. Specifically, normal phase chromatography was performed first to eliminate most of the non-polar materials such as tri and di acyl glycerides and similar entities from the crude mixture. Due to the compatible polar nature between the GTA-446 with other common serum fatty acids and derivatives of medium to long chain lengths, they tend to elute together as a pool of assorted fatty compounds. Consequently, separation and isolation of GTA-446 to obtain >95% purity from compatible components in serum was challenging, and trace impurity levels of fatty acids and their analogues often linger with the purified GTA-C₂₈ fractions even after fine HPLC purification. This made it difficult to obtain a complete structure elucidation using NMR data due to the interfering signals. To overcome this difficulty, a method employing the use of an initial normal phase flash column separation, followed by reverse phase FC prior to injection on an HPLC which eliminated most impurities and leaves GTA-446 and closely-related molecules in one fraction for subsequent HPLC purification was developed.

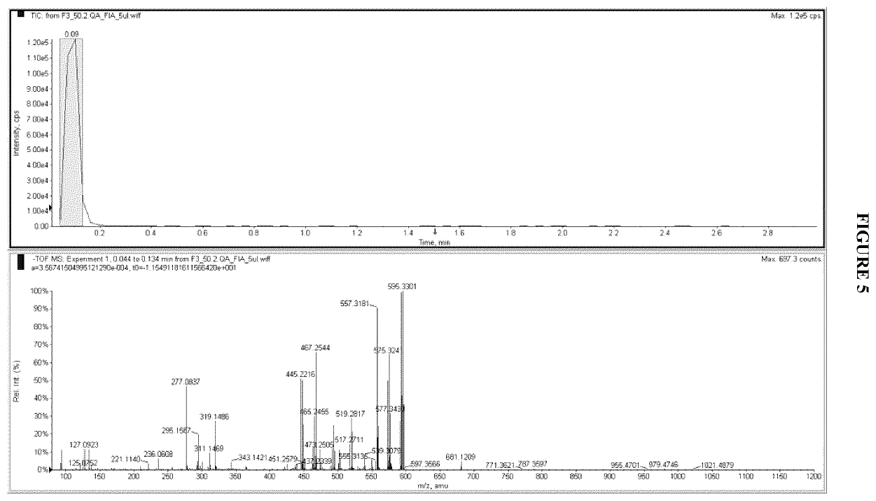

The initial normal phase flash column separation used silica gel and employed a solvent gradient from low to high polarity. Crude serum extracts were loaded onto a silica gel column and eluted with solvents starting at a higher hexane concentration in ethyl acetate, and ending at a higher dichloromethane concentration in methanol. Elutes were collected as six different factions and evaporated to dryness under reduced pressure to obtain the crude samples of each fraction. The dried fractions may be analyzed by time-of-flight or other similar mass spectrometry techniques under negative atmospheric pressure chemical ionization (NAPCI). Full scan flow injection chromatograms showed that GTA-446 biomarkers typically eluted in F3 (20 mg, 2.2% recovery), along with other C₃₆-GTA analogues (m/z: 550-600 amu) with the solvent combination of 60:40 hexane/EtOAc (see FIG. 5). The chromatographic analysis of fractions 1 and 2 in negative APCI mode indicated that most of the fatty acid impurities [m/z: 255.2 (16:0), 279.2 (18:2), 281.2 (18:1), 303.2 (20:4)] were eluted. Analysis of the crude dry downs of fraction 4 indicated the presence of more polar C₃₆-GTAs, with no related molecules eluting after fraction 4.

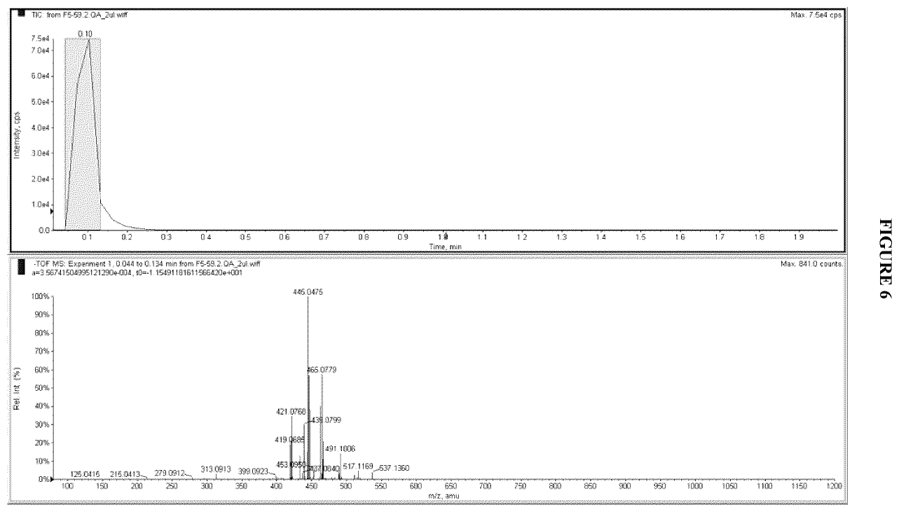

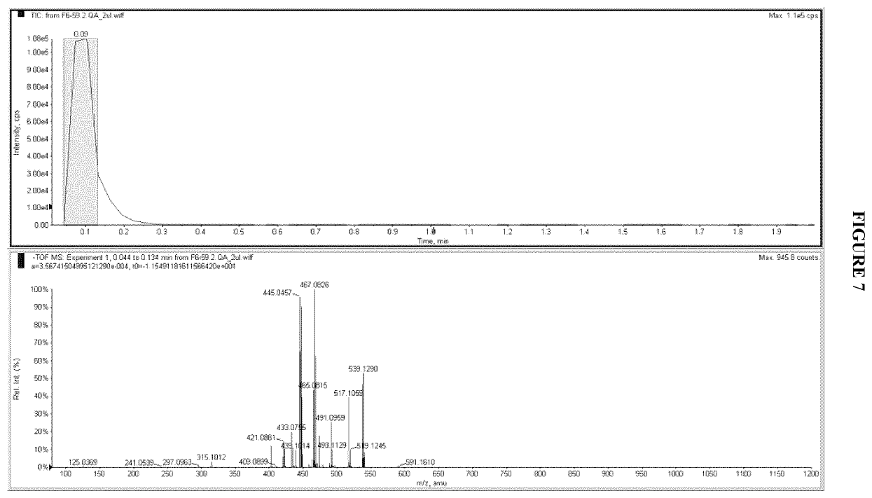

Reverse phase flash column separation relied on C-18 bound silica and started with 60% acetonitrile in water, gradually increasing to 95%, and finishing with 100% methanol wash collecting 15 fractions of 100 ml each. Full scan flow injection chromatograms of the dried fractions was then used to identify fractions containing GTA-446, which typically appear in F5 and 6 along with other C₂₈-GTA analogues with the solvent combinations of 75:25 and 80:20 AcCN:H₂O (FIGS. 6 and 7). Latter fractions F7 and 12 contained a combination of other C₂₈-C₃₆-GTA analogues. GTA-446 rich fractions (F5 and 6) were then combined to obtain another GTA-446 rich fraction containing over 60% GTA-446, but still other C₂₈ and C₃₆-GTA analogues in the sample. To improve the purity further, a tuned, two-step HPLC separation that efficiently resolved GTA-446 structural isomers was performed.

Similar to the FCC separation, the two-step HPLC separation included an initial reverse phase step followed by normal phase (cyano (CN) column) step using prep HPLC. GTA-446 enriched fractions from the reverse phase FCC separation were dissolved in an appropriate solvent (1:1 CH₂Cl₂:CH₃CN) suitable for prep HPLC separation using a reverse phase column with a diode array detector and UV/vis absorption. A gradient elution was then carried out over 35 minutes using two solvent systems made by mixing different ratios of water:acetonitrile:formic acid and collecting 30 second elution fractions between 14-29 minutes. Analysis of the collected fractions showed GTA-446 enrichment to over 75%, but with other C₂₈-GTA analogues as impurities.

GTA-446 enriched fractions from reverse-phase prep HPLC were then pooled and subjected to further purification using a cyano-bound, normal phase Prep HPLC column eluted with an isocratic solvent comprising hexane, EtOAc and formic acid. The UV cut off wavelength was set at a value of λ=>256 nm to overcome signal interferences from EtOAc. Alternatively, the UV detector may be replaced with an MS detector to avoid the spectral interferences of the solvent and small volumes of the eluents may be tested each time for the detection of the presence of GTA-446 using MS data, which may provide a reliable detection method. Analysis of the collected fractions from the second Prep HPLC purification by LCMS typically indicated purity of GTA-446 of >95% of multiple stereosiomeric forms showing similar tandem MS patterns. Depending on initial starting concentration in serum, approximately 5 mg of purified GTA-446 among the various fractions was obtainable from 40 L of human serum.

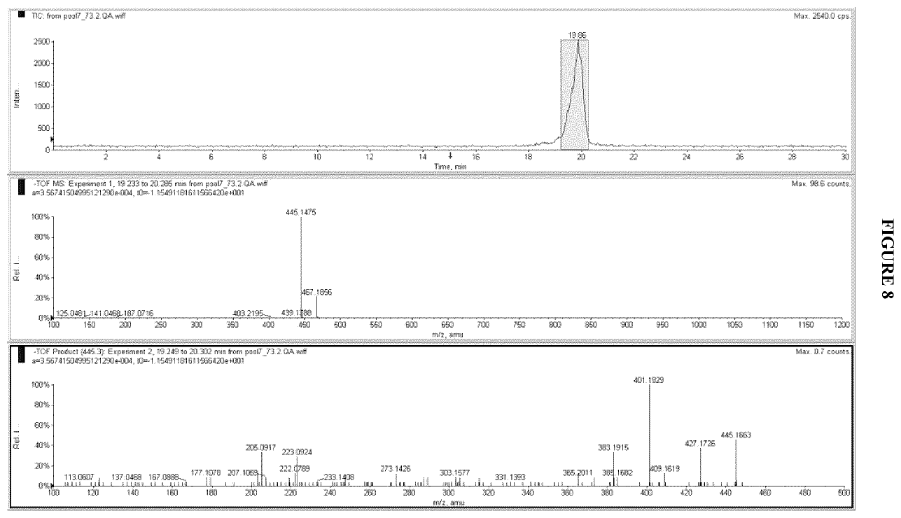

FIG. 8 shows a full scan chromatogram of a GTA-446-rich sample in NAPCI from prep HPLC-RP separation.

### Example 2—Experimental Protocols for a GTA-446 Isolation from Human Serum

In this example, 120 ml of thawed and well mixed Seracare serum was added into a 2000 ml graduated glass bottle. Added slowly into the same bottle was 240 ml of methanol acidified with 0.1% formic acid while swirling the contents. The mixture was allowed to stand for 5 minutes for the completion of the protein precipitation. Added next into the same flask slowly was 480 ml of ethyl acetate solution. The mixture was allowed to stand for a further 5 minutes. The solution was then hand stirred to obtain a homogenize mixture and allocated equally into sixteen 50 ml falcon tubes (˜52.5 ml/tube). These were centrifuged for 10 minutes (3500 rpm/4° C.) to force the precipitate into a tight pallet which sits at the bottom of the tube well separated from the liquids. The supernatant was transferred into a separatory funnel containing 480 ml of hexane well shaken and allowed the phases to be separated. The organic phase was analyzed by mass spectrometry for the presence of GTA-446. The bottom aqueous phase was collected and transferred into a second funnel of hexane for further extraction. The top organic layer was dried under anhydrous sodium sulfate, filtered and dried under reduced pressure to obtain the crude organic extracts of human serum (Total of 208.05 g from processing 45 L of serum).

Total 208 g of crude organic extracts obtained from the above precipitation method was subjected to normal phase flash column chromatography separation. The column conditions and the solvent gradient are summarized in Table 2. Column diameter, weight of silica gel and the volumes of the eluents were scaled up with respect to the weight of the crude organic extracts as used.

TABLE 2
Column Specifications for normal phase flash column
separation of 30 g of crude organic serum extracts
Per 30-40 g of raw organic serum extract

| | |
|---|---|
| Column Inner Diameter | 5 cm |
| Weight of Silica | 100 g (60 A°; 230-400 mesh silica gel) |
| Volume of hexane:EtOAc | 1000 ml (F1, F2) 500 ml each |
| (90:10) | |
| Volume of hexane:EtOAc | 1000 ml (F3-F7) 200 ml each |
| (80:20) | |
| Volume of hexane:EtOAc | 1000 ml (F8-F12) 200 ml each |
| (60:40) | |
| Volume of DCM:MeOH (90:10) | 500 ml (F13) |

The column was mounted using hexane while flashing compressed air to avoid the trapping of air bubbles giving a homogenized fill. 30 g of crude extracts were dissolved in minimum volume of DCM and mounted onto the column. The fractions were collected and based on LCMS analysis of each fraction, those which contained GTAs (F8-F13), were evaporated to dryness under reduced pressure and the dried down were used for further separations. Other fractions were discarded. Total of about 3 g were collected from processing 208 g of crude organic extracts.

Total of 2.5 g of GTA-446 containing pool FCC-NP fractions (from F8-F13 based on the Q-Star data) were further separated in FCC-RP. The column conditions and the solvent gradient are summarized in table 3. Column diameter, weight of silica gel and the volumes of the eluents were scaled up with respect to the weight of the crude organic extracts as used.

TABLE 3
Column Specifications for reverse phase flash column
separation of ~1 g of crude organic serum extracts
Per 1 g of loaded crude FCC_NP dried sample

| | |
|---|---|
| Column Inner Diameter | 3 cm |
| Weight of Silica (C-18) | 20 g |
| Volume of AcCN:H₂O (60:40) | 1000 ml (F1, F2) 500 ml each |
| Volume of AcCN:H₂O (65:35) | 2000 ml (F3-F4) 200 ml each |
| | (F5-F8) 250 ml each |
| Volume of AcCN:H₂O (70:30) | 2000 ml (F9-F16) 250 ml each |
| Volume of AcCN:H₂O (75:25) | 1000 ml (F17-F20) 250 ml each |
| Volume of AcCN:H₂O (80:20) | 1000 ml (F21, F22) 500 ml each |
| Volume of AcCN:H₂O (90:10) | 1000 ml (F23, F24) 500 ml each |
| Volume of AcCN 100% | 1000 ml (F25-F27) ~300 ml each |
| Volume of MeOH 100% | 2000 ml (F28) |

The column was mounted using acetonitrile and equilibrated with 60:40 AcCN:H₂O while flashing compressed air to avoid the trapping of air bubbles giving a homogenized fill. 1 g of crude extracts were dissolved using 1:1 AcCN:DCM and mounted onto the column. The fractions were collected and analyzed by LCMS. Majority of GTA-446 eluted with the combination of AcCN/H₂O 70/30 with a lesser amount eluting in the final fractions using AcCN/H₂O of 65/35. These fractions were dried under reduced pressure, and the weights determined. A total of approximately 1.6 g was collected from processing 2.5 g of crude organic extract. These were subsequently subjected to further purification in HPLC.

The GTA-446 rich pool from FCC_RP (140 mg) was solubilized in 1:1 CH₂Cl₂:ACN to produce a 180 mg/mL solution, which was used as the load for PrepLC purification under reverse phase conditions as follows: Solvent A=H₂O:ACN:Formic acid (95:5:0.05), Solvent B=ACN with 0.05% Formic acid, Flow rate=25.5 mL/min, Temperature=ambient, λ=210 nm Column=21.2 mm×150 mm, 5 μm, PrepHT XDB-C18 (Agilent, 970150-902, SN. USMQ001200) Solvent composition=75% B for 35 minutes Fractionation=30 fractions in total from 14-29 minutes (30 second slices). Individual fractions were pooled based on the UV absorption and retention time windows as shown in table 4.

TABLE 4
Fractions obtained from reverse phase prep-HPLC separation.

| t_{R} window | Vial | Pool | |
|---|---|---|---|
| (min) | number/s | sample name | Wt/mg |
| 14.5-15.5 | 2-5 | Pool 2 | 21.7 |
| 15.5-16.2 | 6 | Pool 3 | 7.8 |
| 16.2-20.0 | 7-12 | Pool 4 | 4.7 |
| 20.0-23.0 | 13-18 | Pool 5 | 11 |
| 23.0-23.5 | 19 | Pool 5B | 1.2 |
| 23.5-25.5 | 20-25 | Pool 6 | 0.9 |
| 25.5-29 | 26-30 | Pool 7 | 6.8 |

7 pooled samples were then analyzed in LCMS using both reverse and normal phase chromatography to visualize the degree of GTA-446 isolation and other possible impurity interferences. Pooled samples 5, 5B and 7 all showed isolated GTA-446 levels. Reverse phase LCMS of pool 5 and 5a showed that the GTA-446 was almost completely isolated but normal phase LCMS spectra showed that further purification in normal phase was needed to resolve between different isomers and other minor impurities.

25.7 mg of reverse phase prep HPLC purified GTA-446 rich sample was dissolved in 150 ul of CH₂Cl₂ and further purified in normal phase prep HPLC (SBCN) using the following LC conditions. Flow rate=4 ml/min, Temperature=ambient, Column=9.4 mm×300 mm, 5 μm, Zorhax SB-CN (Agilent), Injection Volume 40 ul. The LC gradient was as shown in Table 5:

TABLE 5
LC Gradient.

| time (min) | % A (Hexane) | % B (1% FA in EtOAc) |
|---|---|---|
| 0-15 | 95 | 5 |
| 15-20 | 50 | 50 |
| 20-30 | 95 | 5 |

Fractions were pooled based on the retention time windows as shown in Table 6:

TABLE 6
Fractions pooled based on retention time windows.

| t_{R} window (min) | Vial number/s | Pool sample name |
|---|---|---|
| 4.6-5.0 | 10 | Pool 1 |
| 5.0-5.4 | 11, 12 | Pool 2 |
| 5.4-5.8 | 13, 14 | Pool 3 |
| 5.8-6.2 | 15-17 | Pool 4 |
| 6.2-6.8 | 18 | Pool 5 |

Each of the pooled samples were analyzed in LCMS, and showed acceptable purification (>95% GTA-446). Three GTA-446 isomers were identified in pools 1, 2 and 4 at different tR of 2.2, 3.2 and 3.8 minutes respectively. Pool 3 was negligible, and Pool 5 contained mostly the column washings which could be re-purified under the same conditions for more recovery of the target. The pooled samples were dried, weighed and analyzed in NMR spectroscopy for a complete structural elucidation. The final weight for each isomer was as shown in Table 7:

TABLE 7
Final weight for each isomer.

| Pool | tR | Weight/mg |
|---|---|---|
| 1 | 2.2 | 1.8 |
| 2 | 3.2 | 1.4 |
| 4 | 3.8 | 2.3 |

### Example 3—Structural Characterization of GTA-446

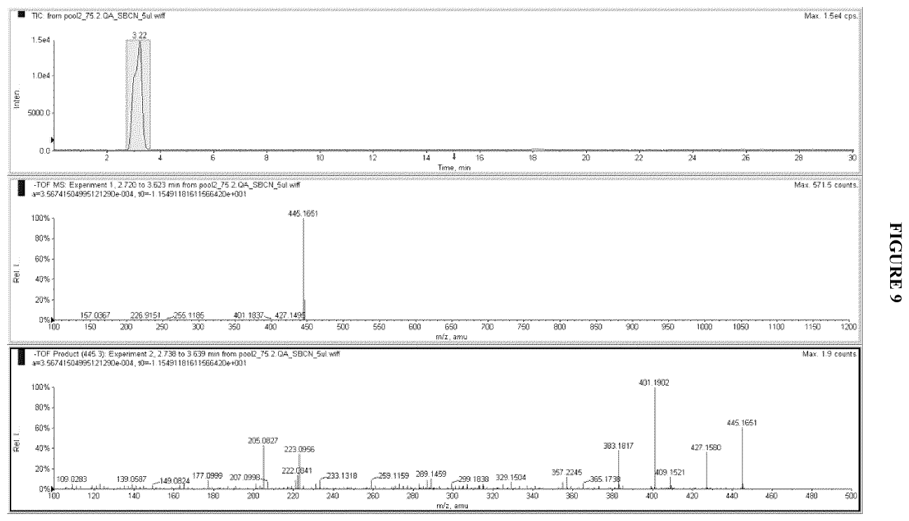

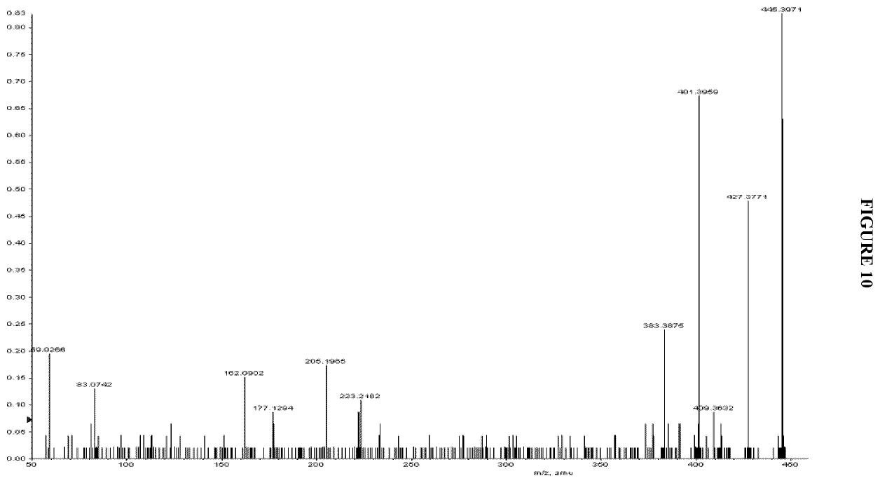

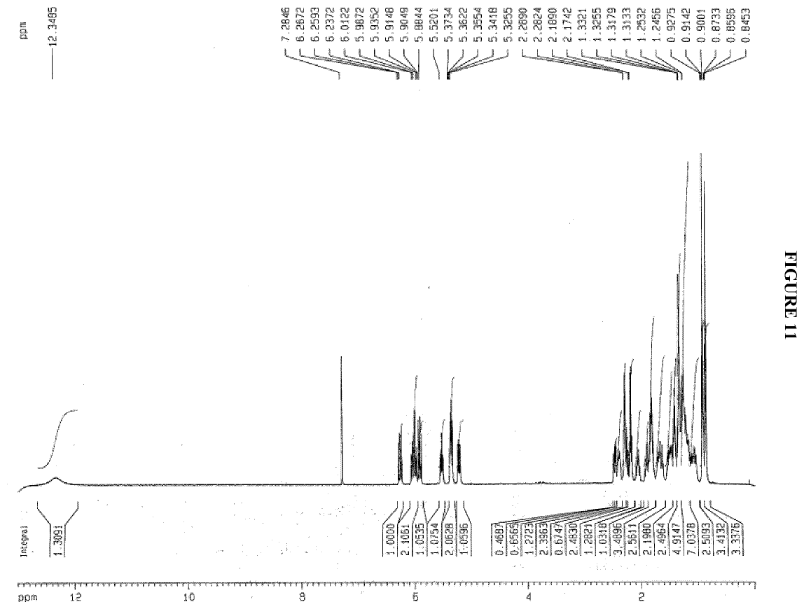

The purified isomers were analyzed in 1D NMR experiments such as ¹H, ¹³C as well as 2D experiments like H—H (COSY), H—C(HMQC) and long range C—H (HMBC) coupling to obtain a complete structural elucidation of GTA-446. The structure of the most abundant isomer was elucidated using the obtained spectral information using MS (FIGS. 9, 10) and NMR spectroscopy (FIGS. 11-14). Evaluation of tandem MS spectra of GTA-446 (m/z 445.3 (M-)) suggest losses of H₂O (M- −18), CO₂ (M- −44), 2×H₂O (M- −36) and CO₂+H₂O (M- −62) at different intensities. This intensity pattern is unique and consistent for MS/MS fragmentation of any serum-derived GTA-446. Loss of CO₂ led to the conclusion that these are possible carboxylic acids. Loss of water initially suggested that they contain hydroxyl groups, and that the loss of water and carbon dioxide together suggested that the carboxyl and hydroxyl functionalities should be separated from each other, rather than from a single carboxyl group. In addition, the MS/MS suggested existence of two hydroxyl groups, while fragment m/z 223 indicated cleavage of the structure into two similar entities, suggesting a possible bridging system between two similar structural components. However, interpretation of a complete structure using MS/MS information only was challenging.

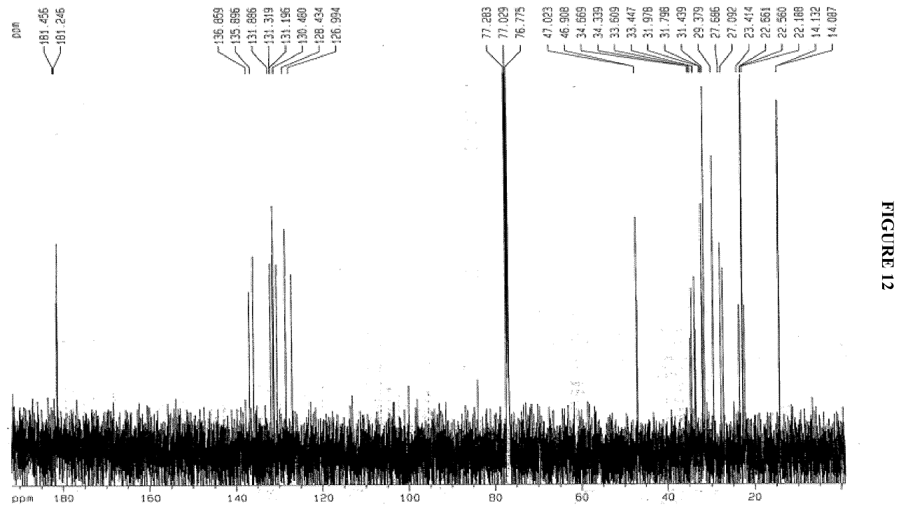

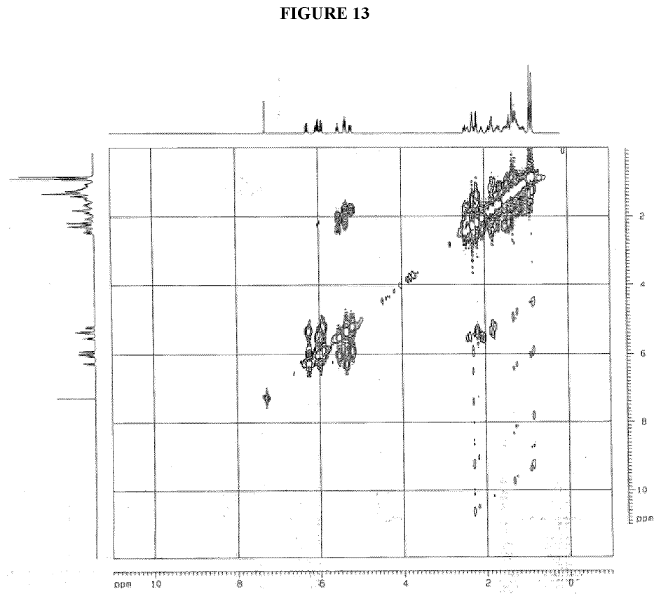

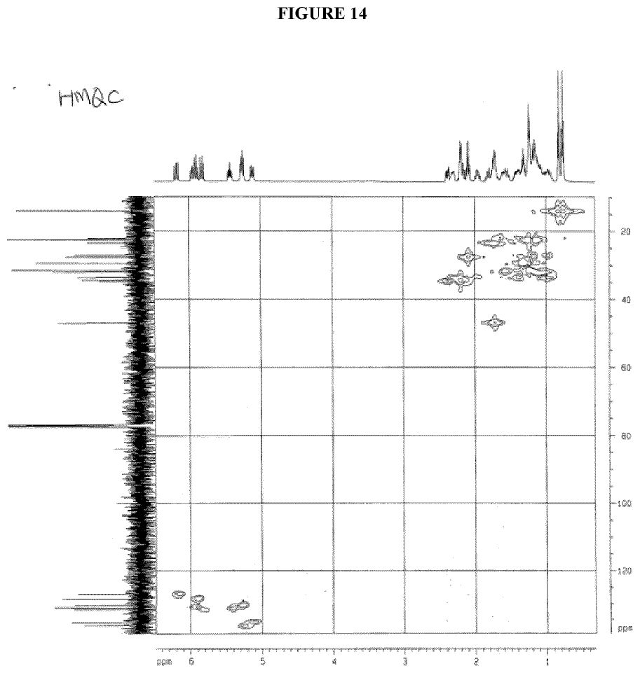

Therefore, ¹H NMR of purified GTA-446 was performed, which showed signals from 8 methylene protons (δ 5.5 to 6.2 pm), two terminal methyl groups (—CH₂CH₃, δ 0.84 and 0.89 pm, each 3H, t), and a broad peak at δ 12.0 ppm from two —COOH groups, and other 34 signals of CH₂ and CH groups from δ 0.98 to 2.48 ppm (see FIG. 11). ¹³C-NMR spectrum showed signals from 8 methylene carbons (δ 126 to 136 pm), two terminal methyl groups (—CH₂CH₃, δ 14.0 and 14.1 pm), and two carbonyl carbons at δ 181.2, 181.4 ppm from two —COOH groups, two methylene carbons at 45.9, 47.0 ppm, 14 secondary carbon signals of CH₂ groups at δ 22.1 to 34.1 ppm (See FIG. 12). ¹H-¹H COSY analysis showed two conjugated systems (FIG. 13). The E,E and E,Z configurations were deduced from their coupling constants. Detailed structure information was obtained from HMQC and HMBC analysis, which clearly showed linkage between two chains at 9- and 5′-position (FIG. 14, 15). Using these spectral information, the structure of GTA-446 was elucidated as 9-(5′-(6′E, 8′Z)-tetradeca-dienoic acid)-(5E, 7E)-tetradeca-dienoic acid ((5E, 7E, 11E, 13Z)-9-pentyl-10-(4′-butanoic acid))-nonadecatetraenoic acid), as shown in formula I:

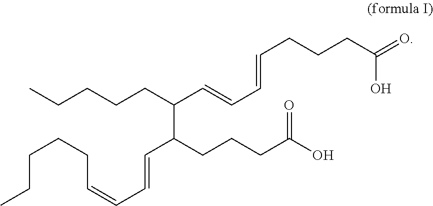 [SMILES: CCCCC/C=C\C=C\C(CCCC(=O)O)C(/C=C/C=C/CCCC(=O)O)CCCCC]

### Example 4—Proposed Synthetic Route for GTA-446

An embodiment of a proposed synthetic route for preparing synthetic GTA-446 and related compounds is described below. It will be understood that this example is intended for the person of skill in the art, and that various modifications, alternatives, additions, deletions, and/or substitutions may be made.

Synthesis of Compound 15 and Compound 16 (as reference standard) may be proposed as follows.

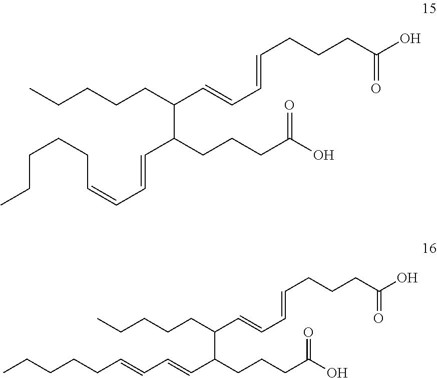 [SMILES: CCCCC/C=C\C=C\C(CCCC(=O)O)C(/C=C/C=C/CCCC(=O)O)CCCCC ; CCCCC/C=C/C=C/C(CCCC(=O)O)C(/C=C/C=C/CCCC(=O)O)CCCCC]

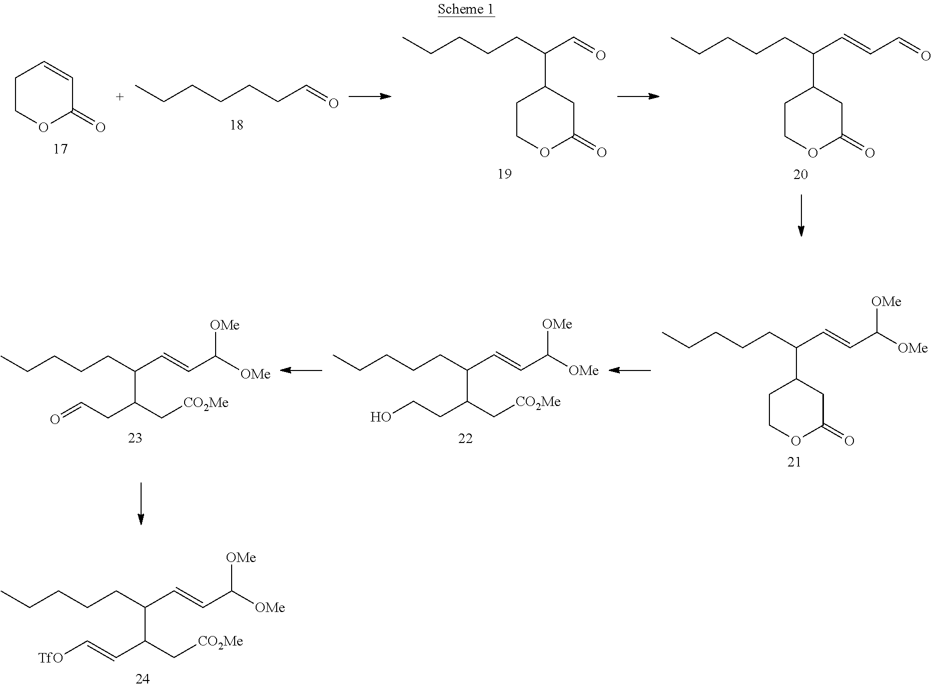

As shown in Scheme 1, a Michael addition between compound 17 and compound 18 may yield compound 19 [1]. A Wittig reaction applied to compound 19 may yield compound 20. Treating compound 20 with methanol [2] or trimethyl orthoformate [3] and acid may yield the dimethyl acetal compound 21. Methanolysis of compound 20 may generate the hydroxyl ester compound 22. Swern oxidation of compound 22 may produce the aldehyde compound 23. Reaction of the aldehyde with triflic anhydride may produce the vinyl triflate compound 24 [4, 5].

As shown in Scheme 2, Sonagashira coupling of compound 24 with compound 25 may yield compound 26 [6]. Subsequent reduction using Lindlar's catalyst may yield the cis-olefin compound 27. Reduction of the methyl ester may yield the alcohol compound 28. Reaction with methanesulfonyl chloride may convert compound 28 to the mesylate compound 29. Displacement of the mesylate with dimethyl malonate may generate compound 30. On treating compound 30 with acid, simultaneous acetal cleavage, ester hydrolysis and decarboxylation may yield the aldehyde compound 31. Final Wittig reaction with, for example, (triphenylphosphoranylidene) acetaldehyde or (4-carboxybutyl)triphenylphosphonium bromide, may complete the synthesis of compound 15.

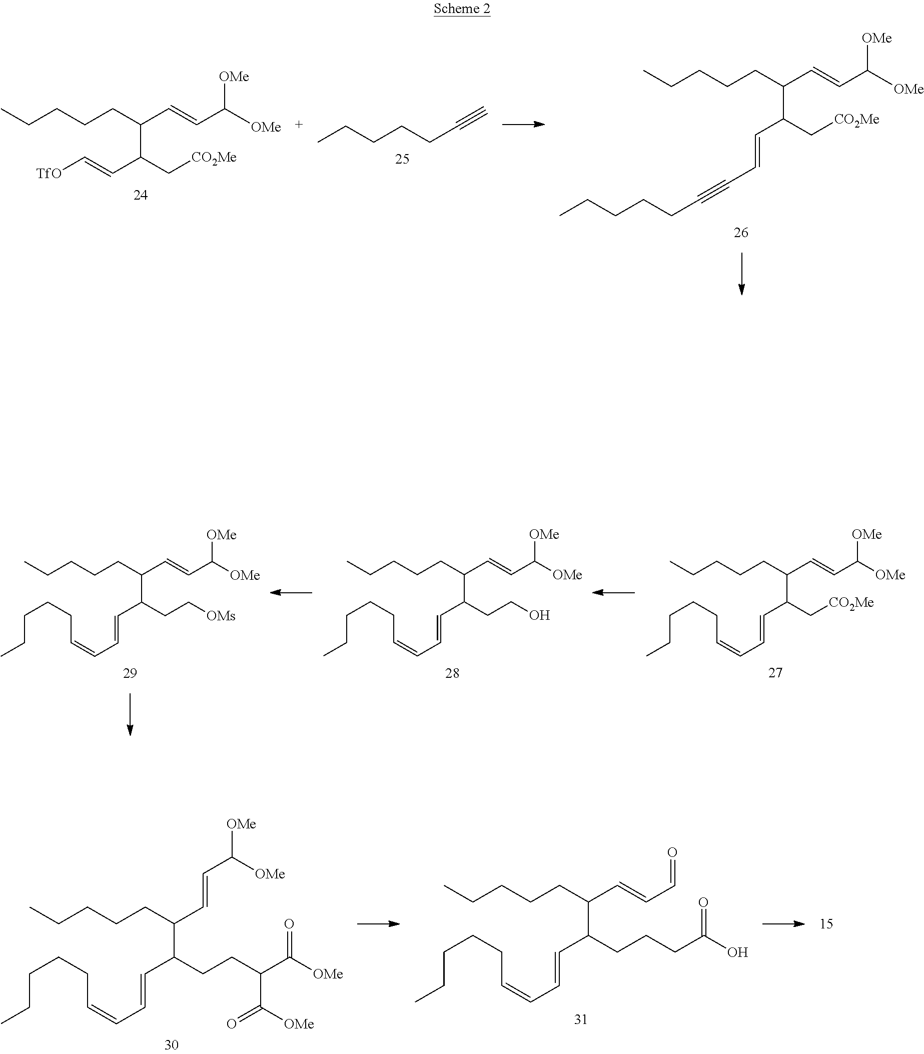

As shown in Scheme 3, Suzuki reaction between compound 24 and compound 32 may yield compound 33 [7]. Completion of compound 16 thus follows the same strategy for conversion of compound 28 to compound 15 as illustrated in Scheme 2.

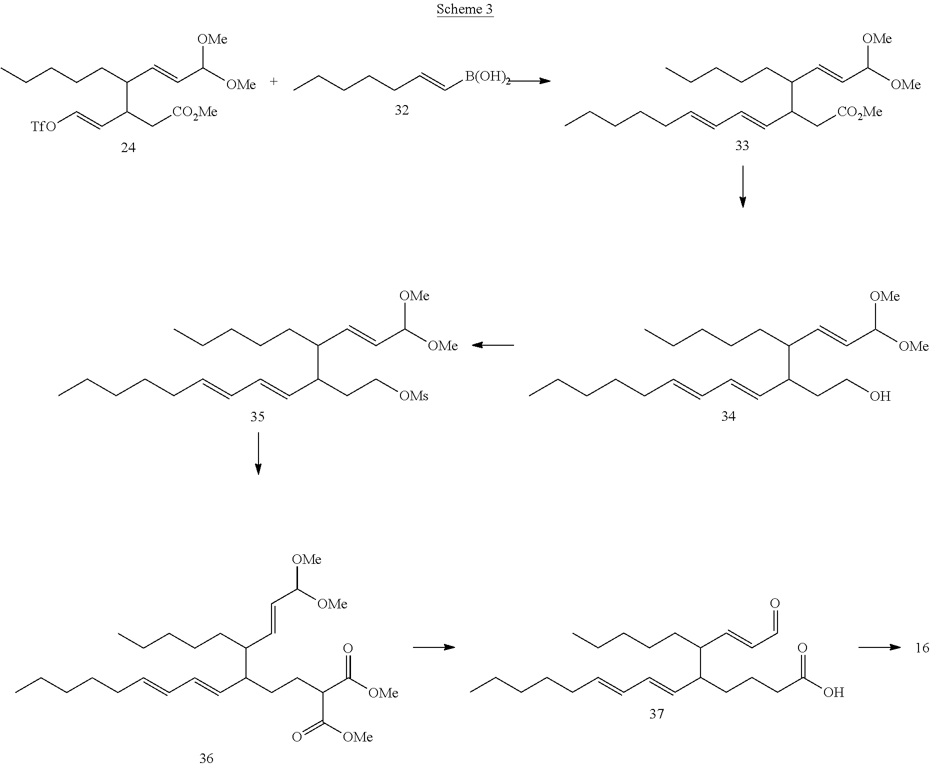

Compound 17 is available from Sigma Aldrich; Compound 18 is available from Sigma Aldrich; Compound 25 is available from Sigma Aldrich; and Compound 32 is available from Sigma Aldrich. All Wittig reagents are available from Sigma Aldrich.

### SYNTHESIS REFERENCES (EACH OF WHICH IS HEREIN INCORPORATED BY REFERENCE IN ITS ENTIRETY)

[1] Schneiderman, Deborah K. and Hillmyer, Marc A.; Aliphatic Polyester Block Polymer Design; Macromolecules, 49(7), 2419-2428; 2016
[2] Andrade, Juan et al; Acetals; Ger. Offen., 3403426, 1 Aug. 1985
[3] Yan, Jingqi et al; Method for preparing acetal by using acraldehyde; Faming Zhuanli Shenqing, 102276427, 14 Dec. 2011
[4] Gracia Martinez, Antonio et al; Synthesis of gem-bistriflates: reaction of aliphatic aldehydes with trifluoromethanesulfonic acid anhydride; Synthesis, (1), 49-51; 1987
[5] Sartori, G. and Maggi, R.; Product subclass 4: synthesis of enol sulfonates; Science of Synthesis, 32, 757-781; 2008
[6] Suffert, Jean and Brueckner, Reinhard; Palladium catalyzed couplings of enol triflates with alkynes under very mild conditions. The stereoselective synthesis of dienediynes from bis(enol triflates); Tetrahedron Letters, 32(11), 1453-6; 1991
[7] Pirovano, Valentina et al; Gold-catalyzed synthesis of tetrahydrocarbazole derivatives through an intermolecular cycloaddition of vinyl indoles and N-allenamides; Chemical Communications, 49(34), 3594-3596; 2013

### Example 5—Additional Proposed Synthetic Route for GTA-446 Intermediate Compound 24

An embodiment of a proposed synthetic route for preparing synthetic GTA-446 and related compounds is described in Example 4, which proceeds through intermediate compound 24. In this example, another embodiment for the preparation of compound 24 is proposed. It will be understood that this example is intended for the person of skill in the art, and that various modifications, alternatives, additions, deletions, and/or substitutions may be made.

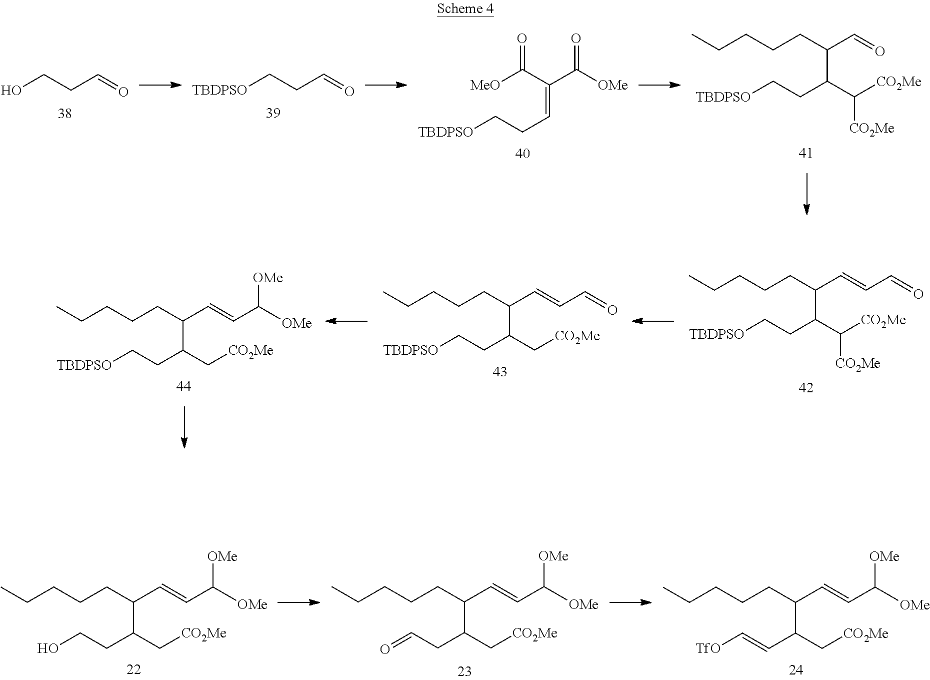

As shown in Scheme 4, 3-hydoxypropionaldehyde compound 38 may be protected as a silyl ether such as a tert-butyl diphenylsilyl (TBDPS) ether giving compound 39. Subsequent aldol condensation with dehydration may yield compound 40. A Michael addition between compound 40 and heptanal (compound 18, Scheme 1) may yield compound 41. A Wittig reaction applied to compound 41 may yield compound 42. Treating compound 42 with DMSO and NaCl may yield compound 43. Reaction of compound 43 with methanol or trimethyl orthoformate and acid may yield the dimethyl acetal compound 44. Reaction of compound 44 with tetrabutylammonium fluoride (TBAF) may yield compound 22. Advancement of compound 22 to compound 24 may proceed as described in Example 4.

One or more illustrative embodiments have been described by way of example. It will be understood to persons skilled in the art that a number of variations and modifications can be made without departing from the scope of the invention as defined in the claims.

### REFERENCES

1. Ritchie S A, Tonita J, Alvi R, et al. Low-serum GTA-446 anti-inflammatory fatty acid levels as a new risk factor for colon cancer. Int J Cancer. 2013; 132:355-362.
2. Ritchie S A, Jayasinghe D, Davies G F, Ahiahonu P, Ma H, Goodenowe D B. Human serum-derived hydroxy long-chain fatty acids exhibit anti-inflammatory and anti-proliferative activity. J Exp Clin Cancer Res. 2011; 30:59.
3. Ritchie S A, Heath D, Yamazaki Y, et al. Reduction of novel circulating long-chain fatty acids in colorectal cancer patients is independent of tumor burden and correlates with age. BMC Gastroenterol. 2010; 10:140.
4. Ritchie S A, Chitou B, Zheng Q, et al. Pancreatic cancer serum biomarker PC-594:Diagnostic performance and comparison to CA19-9. World J Gastroenterol. 2015; 21:6604-6612.
5. Ritchie S A, Akita H, Takemasa I, et al. Metabolic system alterations in pancreatic cancer patient serum: potential for early detection. BMC Cancer. 2013; 13:416.
6. Ritchie S, Heath D, Yamazaki Y, et al. Reduction of novel circulating long-chain fatty acids in colorectal cancer patients is independent of tumor burden and correlates with age. BMC gastroenterology. 2010; 10.
7. Ritchie S, Ahiahonu P, Jayasinghe D, et al. Reduced levels of hydroxylated, polyunsaturated ultra long-chain fatty acids in the serum of colorectal cancer patients: implications for early screening and detection. BMC medicine. 2010; 8.

All references herein and cited elsewhere in this description are hereby incorporated by reference.

What is claimed is:

1. A compound having the structure of formula I or formula III: 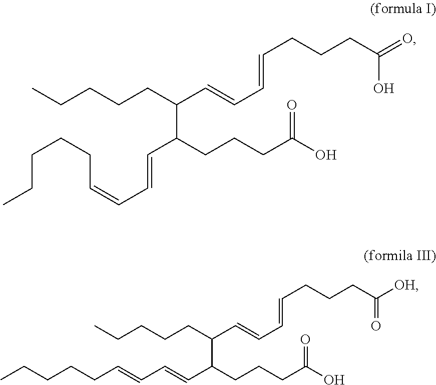 [SMILES: CCCCC/C=C\C=C\C(CCCC(=O)O)C(/C=C/C=C/CCCC(=O)O)CCCCC ; CCCCC/C=C/C=C/C(CCCC(=O)O)C(/C=C/C=C/CCCC(=O)O)CCCCC]
or a salt, ester, prodrug, or labelled derivative thereof.

2. The compound of claim 1, wherein the compound is an isolated compound.

3. The compound of claim 1 or 2, wherein the compound is a synthetically prepared compound.

4. The compound of any one of claims 1-3, wherein the compound is an analytical standard compound.

5. An isotopically labelled compound, the isotopically labelled compound comprising one or more isotopic labels incorporated within the structure of formula I or formula III: 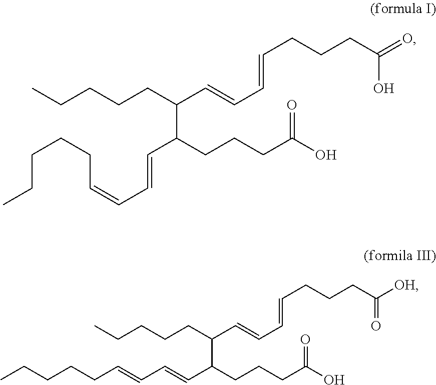 [SMILES: CCCCC/C=C\C=C\C(CCCC(=O)O)C(/C=C/C=C/CCCC(=O)O)CCCCC ; CCCCC/C=C/C=C/C(CCCC(=O)O)C(/C=C/C=C/CCCC(=O)O)CCCCC]
or a salt, ester, or prodrug thereof.

6. The isotopically labelled compound of claim 5, wherein the one or more isotopic labels are stable isotope labels, radioisotope labels, or a combination thereof.

7. The isotopically labelled compound of claim 5 or 6, wherein the one or more isotopic labels are selected from the group consisting of deuterium (²H) and ¹³C.

8. The isotopically labelled compound of claim 5 or 6, wherein the one or more isotopic labels are selected from the group consisting of tritium (³H) and ¹⁴C.

9. The isotopically labelled compound of any one of claims 5-7, wherein the isotopically labelled compound is: 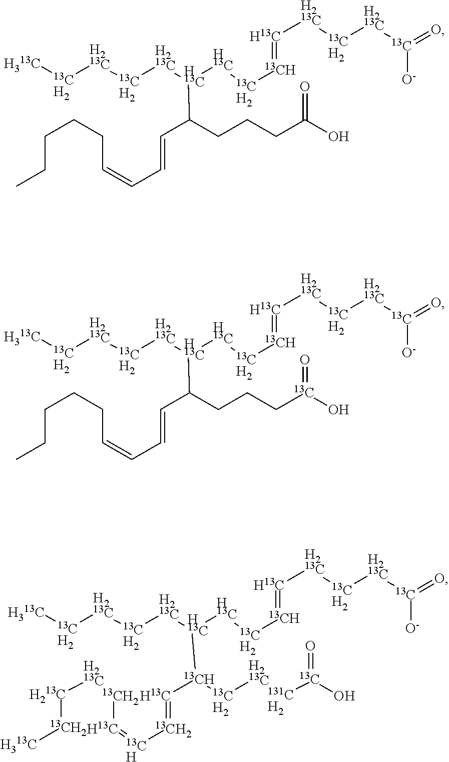
or a derivative thereof in which all carbon-carbon double bonds are in trans configuration. or a salt, ester, or prodrug thereof.

10. The isotopically labelled compound of any one of claims 5-9, wherein the compound is an analytical standard compound.

11. A metabolic tracer composition comprising the isotopically labelled compound of any one of claims 5-9.

12. A composition comprising the compound of any one of claims 1-9, and an excipient, carrier, or diluent.

13. An in vitro or in vivo diagnostic agent comprising the isotopically labelled compound of any one of claims 5-9.

14. A composition comprising the compound of any one of claims 1-9, and an excipient, carrier, or diluent.

15. A method for determining a level of a gastric tract acid (GTA) in a sample, said method comprising:
measuring a GTA detection signal from the sample, the GTA detection signal representative of the GTA level in the sample; and
quantifying the level of the GTA in the sample by comparing the measured GTA detection signal with a calibration reference.

16. The method according to claim 15, wherein the GTA is

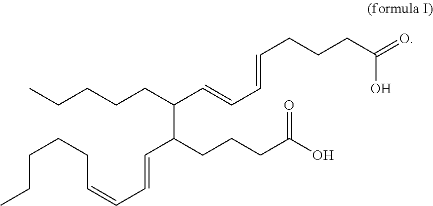 [SMILES: CCCCC/C=C\C=C\C(CCCC(=O)O)C(/C=C/C=C/CCCC(=O)O)CCCCC]

17. The method of claim 15 or 16, wherein the GTA detection signal is measured by mass spectrometry.

18. The method of any one of claims 15-17, wherein the calibration reference comprises a standard curve prepared using known quantities of a compound as defined in any one of claims 5-10.

19. The method of any one of claims 15-17, wherein the calibration reference is obtained by:
spiking the sample with a known quantity of an isotopically labelled compound as defined in any one of claims 5-10; and
measuring an internal standard signal from the sample, the internal standard signal being representative of the known quantity of the isotopically labelled compound spiked into the sample.

20. The method of claim 19, wherein the internal standard signal is measured by mass spectrometry.

21. The method of claim 19 or 20, further comprising a step of determining a ratio of the GTA level in the sample, as represented by the measured GTA detection signal, to the known quantity of isotopically labelled compound spiked into the sample, as represented by the internal standard signal.

22. The method of claim 21, wherein the calibration reference comprises an isotope dilution curve (IDC) generated from a series of mixtures of varying GTA/isotopically labelled compound ratios and concentrations, to which said ratio is compared.

23. The method of claim 22, wherein the IDC is generated from a series of mixtures in which GTA content is varied over a fixed amount of isotopically labelled compound.

24. The method of claim 23, wherein the fixed amount of the isotopically labelled compound is substantially the same as the known quantity of the isotopically labelled compound which is spiked into the sample.

25. Use of the compound of any one of claims 1-10 for determining a level of a gastric tract acid (GTA) in a sample.

26. The use according to claim 25, wherein the GTA is:

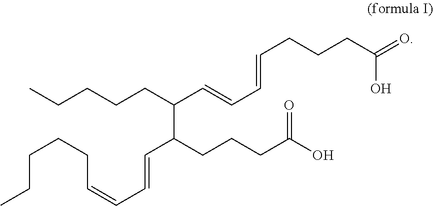 [SMILES: CCCCC/C=C\C=C\C(CCCC(=O)O)C(/C=C/C=C/CCCC(=O)O)CCCCC]

27. Use of the compound of any one of claims 1-10 for generating a calibration reference for use in determining a level of a gastric tract acid (GTA) in a sample.

28. The use according to claim 27, wherein the GTA is:

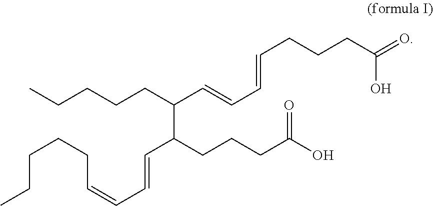 [SMILES: CCCCC/C=C\C=C\C(CCCC(=O)O)C(/C=C/C=C/CCCC(=O)O)CCCCC]

29. Use of the compound of any one of claims 5-9 as an internal standard for use in determining a level of a gastric tract acid (GTA) in a sample.

30. The use according to claim 29, wherein the GTA is:

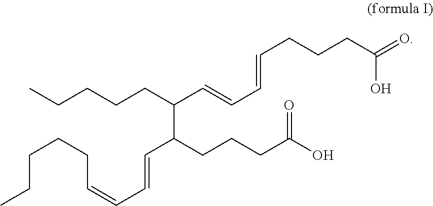 [SMILES: CCCCC/C=C\C=C\C(CCCC(=O)O)C(/C=C/C=C/CCCC(=O)O)CCCCC]

31. A diagnostic method for identifying a subject as having, or being at risk of developing, colorectal cancer, said method comprising:
determining a level of a gastric tract acid (GTA) in a sample obtained from the subject by
measuring a GTA detection signal from the sample, the GTA detection signal representative of the GTA level in the sample; and
quantifying the level of the GTA in the sample by comparing the measured GTA detection signal with a calibration reference, and
identifying the subject as having, or being at risk of developing, colorectal cancer when the determined level of the GTA in the sample is reduced in comparison to a healthy control group,
wherein the GTA is:

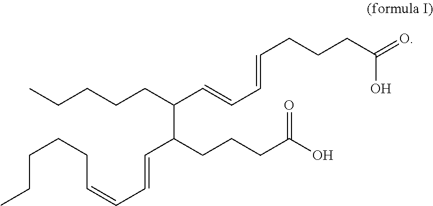 [SMILES: CCCCC/C=C\C=C\C(CCCC(=O)O)C(/C=C/C=C/CCCC(=O)O)CCCCC]

32. The method of claim 31, wherein the GTA detection signal is measured by mass spectrometry.

33. The method of claim 31 or 32, wherein the calibration reference comprises a standard curve prepared using known quantities of a compound as defined in any one of claims 5-10.

34. The method of claim 31 or 32, wherein the step of determining the level of the GTA in the sample obtained from the subject comprises:
spiking the sample with a known quantity of an isotopically labelled compound as defined in any one of claims 5-10; and
measuring an internal standard signal from the sample, the internal standard signal being representative of the known quantity of the isotopically labelled compound spiked into the sample.

35. The method of claim 34, wherein the internal standard signal is measured by mass spectrometry.

36. The method of claim 34 or 35, further comprising a step of determining a ratio of the GTA level in the sample, as represented by the measured GTA detection signal, to the known quantity of isotopically labelled compound spiked into the sample, as represented by the internal standard signal.

37. The method of claim 36, wherein the calibration reference comprises an isotope dilution curve (IDC) generated from a series of mixtures of varying GTA/isotopically labelled compound ratios and concentrations, to which said ratio is compared.

38. The method of claim 37, wherein the IDC is generated from a series of mixtures in which GTA content is varied over a fixed amount of isotopically labelled compound.

39. The method of claim 38, wherein the fixed amount of the isotopically labelled compound is substantially the same as the known quantity of the isotopically labelled compound which is spiked into the sample.

40. Use of the compound of any one of claims 1-10 in a diagnostic method for identifying a subject as having, or being at risk of developing, a colorectal cancer associated with altered levels of a gastric tract acid (GTA) which is:

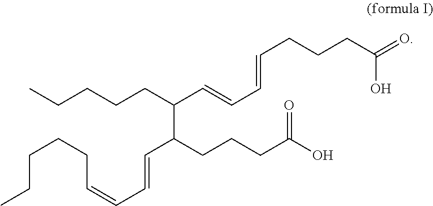 [SMILES: CCCCC/C=C\C=C\C(CCCC(=O)O)C(/C=C/C=C/CCCC(=O)O)CCCCC]

41. Use of the compound of any one of claims 1-10 for generating a calibration reference for use in a diagnostic method for identifying a subject as having, or being at risk of developing, a colorectal cancer associated with altered levels of a gastric tract acid (GTA) which is:

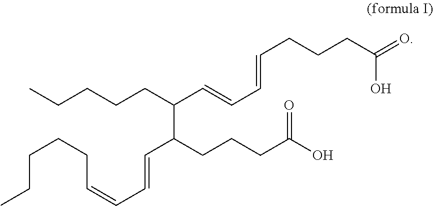 [SMILES: CCCCC/C=C\C=C\C(CCCC(=O)O)C(/C=C/C=C/CCCC(=O)O)CCCCC]

42. Use of the compound of any one of claims 5-9 as an internal standard for use in a diagnostic method for identifying a subject as having, or being at risk of developing, a colorectal cancer associated with altered levels of a gastric tract acid (GTA) which is:

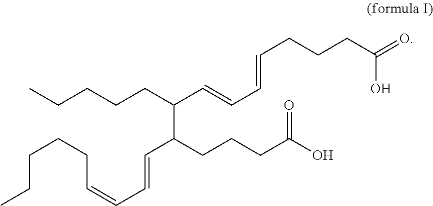 [SMILES: CCCCC/C=C\C=C\C(CCCC(=O)O)C(/C=C/C=C/CCCC(=O)O)CCCCC]

43. An antibody, or antigen-binding fragment thereof, which specifically binds a compound of formula I:

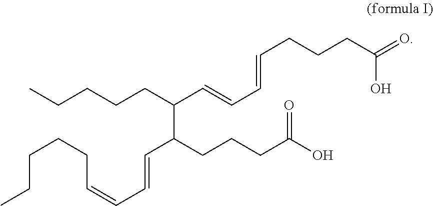 [SMILES: CCCCC/C=C\C=C\C(CCCC(=O)O)C(/C=C/C=C/CCCC(=O)O)CCCCC]

44. The antibody, or antigen-binding fragment thereof, of claim 43, wherein the antibody is a monoclonal or a polyclonal antibody.

45. Use of a compound according to any one of claims 1-3 as an antigen for preparing an antibody which specifically binds to an antigenic epitope of the compound.

46. Use of the antibody of claim 43 or 44 for detecting or quantifying a level of a gastric tract acid (GTA) in a sample by immunoassay, wherein the GTA is:

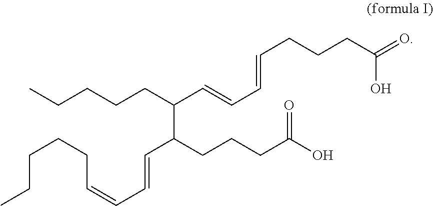 [SMILES: CCCCC/C=C\C=C\C(CCCC(=O)O)C(/C=C/C=C/CCCC(=O)O)CCCCC]

47. Use of the antibody of claim 43 or 44 in a diagnostic method for identifying a subject as having, or being at risk of developing, a colorectal cancer associated with altered levels of a gastric tract acid (GTA) which is:

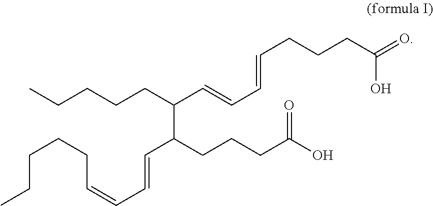 [SMILES: CCCCC/C=C\C=C\C(CCCC(=O)O)C(/C=C/C=C/CCCC(=O)O)CCCCC]

48. A method for determining a level of a gastric tract acid (GTA) in a sample, said method comprising:
measuring a level of the GTA in the sample using an immunoassay employing an antibody, or antigen-binding fragment thereof, which specifically binds to the GTA;
wherein the GTA is:

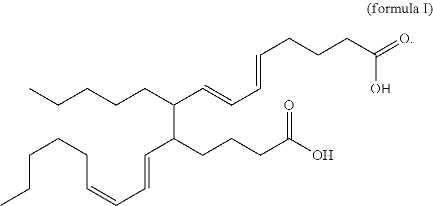 [SMILES: CCCCC/C=C\C=C\C(CCCC(=O)O)C(/C=C/C=C/CCCC(=O)O)CCCCC]

49. The method of claim 48, wherein the immunoassay comprises an enzyme-linked immunosorbent assay (ELISA).

50. The method of claim 48 or 49, further comprising a step of using a control sample comprising a compound as defined in any one of claims 1-10 as a positive control in the immunoassay.

51. The method of any one of claims 48-49, further comprising a step of using a standard curve to extrapolate the level of the GTA in the sample, the standard curve having been generated using a plurality of known quantities of a compound as defined in any one of claims 1-10.

52. A diagnostic method for identifying a subject as having, or being at risk of developing, colorectal cancer, said method comprising:
determining a level of a gastric tract acid (GTA) in a sample obtained from the subject by
measuring a level of the GTA in the sample using an immunoassay employing an antibody, or antigen-binding fragment thereof, which specifically binds to the GTA; and
identifying the subject as having, or being at risk of developing, colorectal cancer when the determined level of the GTA in the sample is reduced in comparison to a healthy control group,
wherein the GTA is:

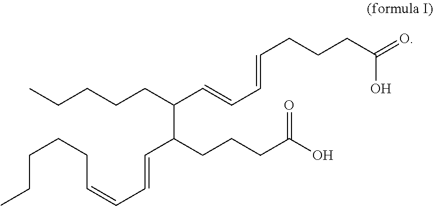 [SMILES: CCCCC/C=C\C=C\C(CCCC(=O)O)C(/C=C/C=C/CCCC(=O)O)CCCCC]

53. The method of claim 52, wherein the immunoassay comprises an enzyme-linked immunosorbent assay (ELISA).

54. The method of claim 52 or 53, further comprising a step of using a control sample comprising a compound as defined in any one of claims 1-10 as a positive control in the immunoassay.

55. The method of any one of claims 52-54, wherein the step of determining the level of the GTA in the sample further comprises using a standard curve to extrapolate the level of the GTA in the sample, the standard curve having been generated using a plurality of known quantities of a compound as defined in any one of claims 1-10.

56. A kit for quantifying a level of a gastric tract acid (GTA) in a sample, the kit comprising at least one of:
a compound according to any one of claims 1-10;
a metabolic tracer according to claim 11;
a composition according to claim 12 or 14;
a diagnostic agent according to claim 13; and
an antibody according to claim 43 or 44;
and, optionally, further comprising a set of instructions for performing a method as defined in any one of claims 15-24 and 48-51.

57. A diagnostic kit for identifying a subject as having, or being at risk of developing, colorectal cancer, the kit comprising at least one of:
a compound according to any one of claims 1-10;
a metabolic tracer according to claim 11;
a composition according to claim 12 or 14;
a diagnostic agent according to claim 13; and
an antibody according to claim 43 or 44;
and, optionally, further comprising a set of instructions for performing a method as defined in any one of claims 31-39 and 52-55.

58. A compound having the formula: 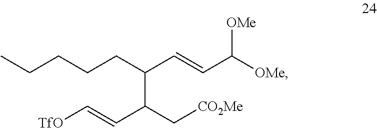 [SMILES: CCCCCC(/C=C/C(OC)OC)C(/C=C/OS(=O)(=O)C(F)(F)F)CC(=O)OC]
or a labelled derivative thereof.

59. Use of the compound of claim 58 in the synthesis of a compound having the formula: 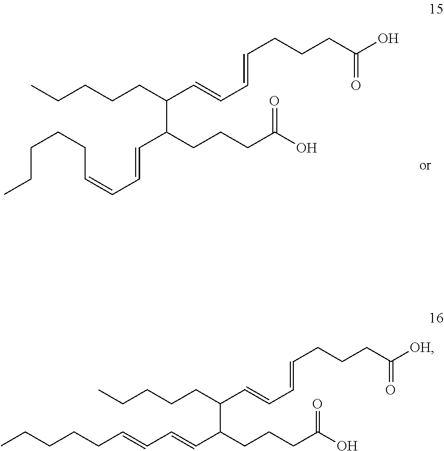 [SMILES: CCCCC/C=C\C=C\C(CCCC(=O)O)C(/C=C/C=C/CCCC(=O)O)CCCCC ; CCCCC/C=C/C=C/C(CCCC(=O)O)C(/C=C/C=C/CCCC(=O)O)CCCCC]
or a salt, ester, prodrug, or labelled derivative thereof.

60. A method for synthesizing a compound having formula (I): 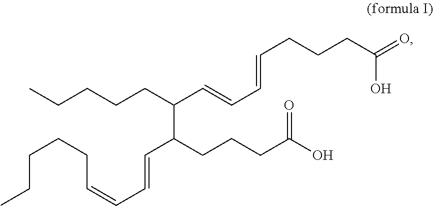 [SMILES: CCCCC/C=C\C=C\C(CCCC(=O)O)C(/C=C/C=C/CCCC(=O)O)CCCCC]
or an isotopically labelled derivative thereof,
said method comprising:
providing a compound according to claim 58;
performing a Sonagashira coupling of the compound with 1-heptyne;
performing a reduction with Lindlar's catalyst;
performing a methyl ester reduction;
performing a reaction with methanesulfonyl chloride;
performing a mesylate displacement with dimethyl malonate;
performing an acid treatment for simultaneous acetal cleavage, ester hydrolysis, and decarboxylation; and
performing a Wittig reaction to yield the compound of formula I, or an isotopically labelled derivative thereof,
wherein the compound according to claim 58, or at least one reactant in the method, comprises at least one isotopically labelled atom which is incorporated into the resulting compound of formula I when an isotopically labelled derivative of formula I is synthesized.

61. The method according to claim 60, wherein the Wittig reaction comprises reaction with (triphenylphosphoranylidene) acetaldehyde or (4-carboxybutyl)triphenylphosphonium bromide.

62. A compound having formula D: 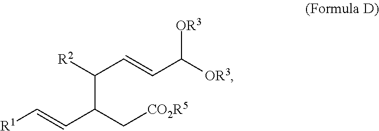
wherein:
R¹ is —Sn(R¹⁰)₃, —OTf, —Cl, —Br, —I, —B(OH)₂ or 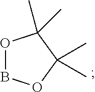
R² is optionally substituted saturated or unsaturated C₁-C₂₀ alkyl, saturated or unsaturated C₂-C₂₀ alkenyl, or saturated or unsaturated C₂-C₂₀ alkynyl;
each R³ is, independently, optionally substituted C₁-C₆ alkyl, or the R³ groups together form an optionally substituted ethylene or propylene group bridging the attached oxygen atoms to form a five- or six-membered ring;
R⁵ is optionally substituted C₁-C₆ alkyl; and
R¹⁰ is optionally substituted C₁-C₆ alkyl,
or a labelled derivative thereof.

63. Use of the compound of claim 62 in the synthesis of a gastric tract acid (GTA), or a derivative thereof.

64. The use of claim 63, wherein the GTA or the derivative thereof is a compound of formula N or S: 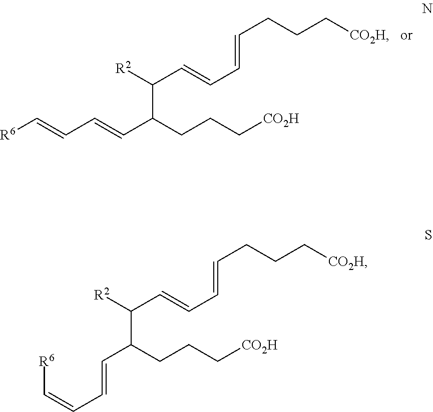
or a salt, ester, prodrug, or labelled derivative thereof,
wherein
R² is optionally substituted saturated or unsaturated C₁-C₂₀ alkyl, saturated or unsaturated C₂-C₂₀ alkenyl, or saturated or unsaturated C₂-C₂₀ alkynyl; and
R⁶ is optionally substituted saturated or unsaturated C₁-C₂₀ alkyl, saturated or unsaturated C₂-C₂₀ alkenyl, or saturated or unsaturated C₂-C₂₀ alkynyl.

65. A method for synthesizing a compound of formula N or S as defined in claim 64, or an isotopically labelled derivative thereof, said method comprising:
providing a compound according to claim 62;
performing a coupling reaction and, optionally, a reduction, to replace the R¹ group with an optionally substituted saturated or unsaturated alkyl, saturated or unsaturated alkenyl, or saturated or unsaturated alkynyl;
converting the R⁵-containing ester to a hydroxyl group;
converting the hydroxyl group to a leaving group;
displacing the leaving group with a dialkyl malonate;
performing acetal hydrolysis, ester hydrolysis, and decarboxylation, forming an aldehyde; and
performing a coupling reaction at the aldehyde to yield the compound of formula N or S, or an isotopically labelled derivative thereof,
wherein the compound according to claim 62, or at least one reactant in the method, comprises at least one isotopically labelled atom which is incorporated into the resulting compound of formula N or S when an isotopically labelled derivative of formula N or S is synthesized.

66. The compound of any one of claims 1-10, wherein the compound is: 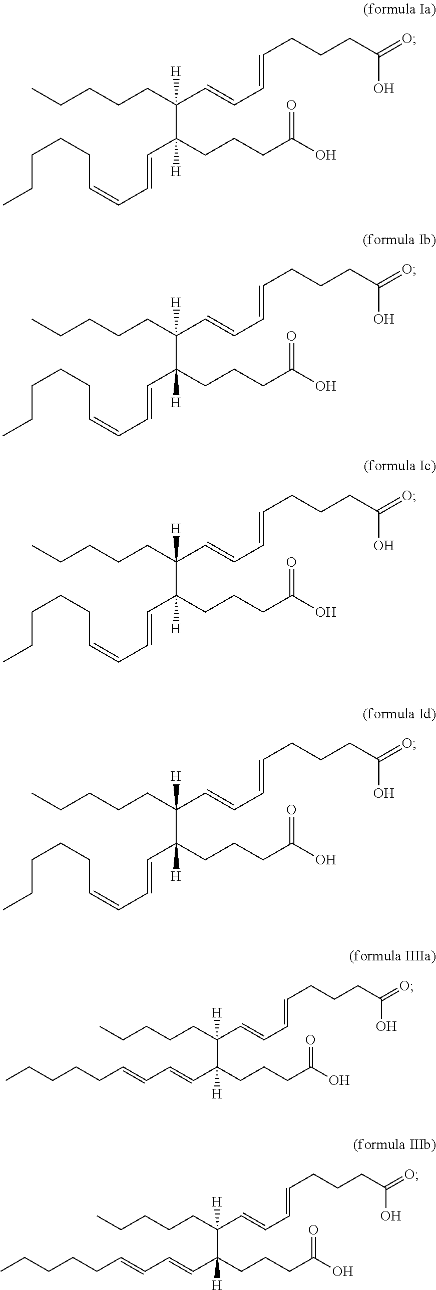 [SMILES: CCCCC/C=C\C=C\[C@H](CCCC(=O)O)[C@H](/C=C/C=C/CCCC(=O)O)CCCCC ; CCCCC/C=C\C=C\[C@@H](CCCC(=O)O)[C@H](/C=C/C=C/CCCC(=O)O)CCCCC ; CCCCC/C=C\C=C\[C@H](CCCC(=O)O)[C@@H](/C=C/C=C/CCCC(=O)O)CCCCC ; CCCCC/C=C\C=C\[C@@H](CCCC(=O)O)[C@@H](/C=C/C=C/CCCC(=O)O)CCCCC ; CCCCC/C=C/C=C/[C@H](CCCC(=O)O)[C@H](/C=C/C=C/CCCC(=O)O)CCCCC ; CCCCC/C=C/C=C/[C@@H](CCCC(=O)O)[C@H](/C=C/C=C/CCCC(=O)O)CCCCC] 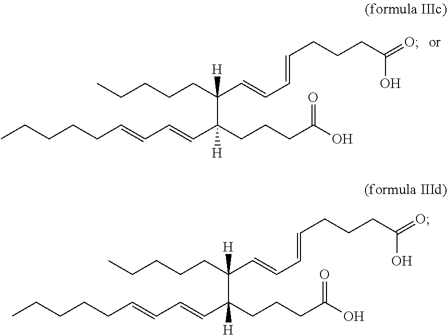 [SMILES: CCCCC/C=C/C=C/[C@H](CCCC(=O)O)[C@@H](/C=C/C=C/CCCC(=O)O)CCCCC ; CCCCC/C=C/C=C/[C@@H](CCCC(=O)O)[C@@H](/C=C/C=C/CCCC(=O)O)CCCCC]
or any combination thereof;
or a salt, ester, prodrug, or labelled derivative thereof.

67. A compound or method as described herein.
